SEQUENCE LISTING (5 sequences)

SEQ ID NO: 1 - protein, 12 aa, Vigna angularis
VAEQEQEQAKRQ

SEQ ID NO: 2 - protein, 10 aa, Vigna angularis
LPQQEQGHDR

SEQ ID NO: 3 - protein, 10 aa, Vigna angularis
VDAQPEEQQR

SEQ ID NO: 4 - protein, 10 aa, Vigna angularis
GINAENNQRN

SEQ ID NO: 5 - protein, 9 aa, Vigna angularis
EYTGDAAGR

(19) United States
(12) Patent Application Publication
(10) Pub. No.: US 2026/0327274 A1
(43) Pub. Date: Oct. 22, 2026

(54) PROTEIN HYDROLYSATES OF AZUKI BEANS AND COSMETIC USES THEREOF

(71) Applicant: LUBRIZOL ADVANCED MATERIALS, INC., Cleveland, OH (US)

(72) Inventors: Ramon SUBIRÓS, Gava, Barcelona (ES); Albert SOLEY ASTALS, Sabadell, Barcelona (ES); Núria ALMINANA DOMENÈCH, Baixos, Barcelona (ES); Cristina BONELL, Gava, Barcelona (ES); Consuelo GARCIA HERNÁNDEZ, Gava, Barcelona (ES); Catrin YOUSSIF, Barcelona (ES); Sandra MÉNDEZ, Badalona (ES); Néstor IBARZ BLANCH, Fraga, Huesca (ES); Begoña MUGUERZA MARQUÍNEZ, Valencia (ES); Francisca Isabel BRAVO VÁZQUEZ, Badajoz (ES)

(73) Assignee: LUBRIZOL ADVANCED MATERIALS, INC., Cleveland, OH (US)

(21) Appl. No.: 18/847,390

(22) PCT Filed: Mar. 15, 2023
(86) PCT No.: PCT/IB2023/052524

§ 371 Date: Sep. 16, 2024

(30) Foreign Application Priority Data
Mar. 16, 2022 (EP) 22382248.7
Mar. 15, 2023 (WO) PCT/IB2023/052524

(51) Int. Cl. C07K 14/415; A61K 8/64; A61Q 5/00; A61Q 17/04; A61Q 19/00; A61Q 19/08; C12N 9/54; C12P 21/06

(52) U.S. Cl. CPC C07K 14/415; A61K 8/645; A61Q 5/002; A61Q 17/04; A61Q 19/00; A61Q 19/08; C12N 9/54; C12P 21/06; A61K 2800/522; C12Y 304/21062

(57) ABSTRACT

Protein hydrolysate obtained from azuki beans and its use, in particular, in the cosmetic treatment and/or care of the skin and/or hair. The protein hydrolysate comprises at least one compound chosen from: (i) Val-Ala-Glu-GIn-Glu-GIn-Glu-GIn-Ala-Lys-Arg-GIn (SEQ ID No. 1); (ii) Leu-Pro-GIn-GIn-Glu-GIn-Gly-His-Asp-Arg (SEQ ID No. 2); (iii) Val-Asp-Ala-GIn-Pro-Glu-Glu-GIn-GIn-Arg (SEQ ID No. 3); and (iv) Gly-Ile-Asn-Ala-Glu-Asn-Asn-GIn-Arg-Asn (SEQ ID No. 4). A method for preparing the protein hydrolysate is also disclosed.

This application claims the priority of International Application PCT/IB2023/052524, filed Mar. 15, 2023, and EP 22382248.7, filed Mar. 16, 2022, from which the PCT application claims priority, the disclosures of which are incorporated herein in their entireties by reference.

### FIELD OF THE INVENTION

The invention relates to protein hydrolysates obtained from azuki beans and their use, in particular, in the cosmetic treatment and/or care of the skin and/or hair. The protein hydrolysates are useful in protecting the skin and/or hair against adverse effects of the exposome, including adverse effects arising from air pollution and/or sunlight. Further, the protein hydrolysates are useful in protecting hair against chemical and heat stress, for example, chemical and heat stress arising from personal care regimes. Methods of preparing the protein hydrolysates are also disclosed.

### BACKGROUND OF THE INVENTION

The totality of extrinsic factors to which an individual is exposed over a lifetime is referred to as the exposome. The appearance of skin, the appearance of hair, and aging are influenced by the combined action of intrinsic and extrinsic factors. Intrinsic factors that negatively impact skin and hair include, for example, chronological aging, a person's genetic makeup, and other biological changes that occur from within the skin. For decades, external damaging factors on the skin and hair have been ignored. However, there is growing evidence that the exposome can have an adverse effect on skin and hair appearance [Vierkötter, A, et al., “Airborne particle exposure and extrinsic skin aging,” J. Invest. Dermatol. 2010; and Parrado, C. et al., “Environmental Stressors on Skin Aging. Mechanistic insights.” Front. Pharmacol. 2019; 10:759, published 9 Jul. 2019, doi:10.3389/fphar.2019.00759].

Examples of extrinsic factors associated with the exposome include exposure to ultraviolet (UV) rays emanating from the sun, high energy visible light (violet-blue) emitted by devices such as TV/computer/smartphone screens, as well as harmful chemical agents found in airborne pollution such as smog and cigarette smoke.

Ultraviolet (UV) radiation is one of the main external factors that has a detrimental effect on the appearance of skin and hair. Extrinsic aging has been regarded to be mainly the result of sun radiation exposure, also known as photoaging.

Following in-depth study of the effects of pollution on health, awareness about the deleterious effects of pollution on the appearance of skin and hair has increased [Vierkötter, A., et al., “Airborne particle exposure and extrinsic skin aging.” J. Invest. Dermatol. 2010]. Additionally, it is known that the adverse effects of various pollutants on the skin and hair can be amplified due to their interaction with UV radiation [Drakaki, E., et al., “Air pollution and the skin.” Front. Environ. Sci., 2014]. For example, it is well known that the main role of the skin is to form a barrier between the host and the environment. The skin must protect an organism from chemicals, ultraviolet light, mechanical damage, and pathogenic microorganisms. Also, the skin must provide an efficient permeability barrier that prevents the loss of water and electrolytes. [Feingold, K., Elias, P., “The important role of lipids in the epidermis and their role in the formation and maintenance of the cutaneous barrier.” Biochim. Biophys. Acta. 2014] Air pollutants can have major effects on the function of the skin. Alterations that disturb the skin barrier function, in either stratum corneum lipid metabolism or protein components of the corneocytes, adversely affect skin appearance. Air pollutants may induce severe interference of normal functions of lipids, DNA and/or proteins of the human skin via oxidative damage [Eleni Drakaki, Clio Dessinioti and Christina V. Antoniou, “Air pollution and the skin,” Front. Environ. Sci., 2014].

Furthermore, continued anthropogenic emissions of greenhouse gases (mainly carbon dioxide) have resulted in an increase of the Earth's temperature by more than 1° C. since 1880, with the majority of warming occurring in the last 45 years [National Aeronautics and Space Administration. NASA ozone watch. 2020b [cited 2020 Apr. 4]. Therefore, although solar radiation and pollutants are two important components of the exposome, other environmental components such as greenhouse gases and temperature (that affects all biochemical reactions) are also highly relevant.

The above-mentioned extrinsic factors can lead to high production of reactive oxidative species (ROS). In the skin, the production of ROS can affect the skin's defenses by quickly depleting the enzymatic (glutathione peroxidase, glutathione reductase, superoxide dismutase, catalase) and nonenzymatic (vitamin E, vitamin C, and glutathione) antioxidant capacity [Valacchi, G., et al., “Cutaneous responses to environmental stressors,” Ann. N Y Acad. Sci. 2012]. In hair, oxidative stress can also have significant negative effects. Proteins are the principal components of hair shafts (65% to 95%) and protein carbonylation, which is an irreversible oxidative protein modification that can significantly damage hair. The protection of hair proteins from carbonylation due to pollutants is desirable for hair and scalp protection against urban stress. [Baraibar, Martin, “Urban pollution induces irreversible carbonylation of hair proteins,” Conference: IFSCC, 2018].

In addition, skin and, in particular, hair are also subjected to other stressors from daily personal care routines. Diverse causes of extrinsic damage to the hair shaft can be roughly divided into physical and chemical causes. The human hair shaft consists of the cortex with a central axial medulla and an external cuticular layer. Chemical causes of hair damage include bleaching and hair dyeing, while physical causes include thermal treatments (such as treatments with straightening irons), friction from hair accessories and washing [Lee, Y., et al., “Comparison of hair shaft damage after chemical treatment in Asian, White European, and African hair,” Int. J. Dermatol. 2014].

The effects of external factors on skin and hair can be distinguished from those associated with intrinsic factors. Skin that is adversely affected by the exposome may show more symptoms of aging such as coarse wrinkles. Hair adversely affected by the exposome can look damaged and be less manageable.

From a preventive point of view, counteracting exposome factors (i.e., extrinsic factors) is of particular interest because extrinsic factors can be modified more easily than intrinsic factors.

It is desirable for the skin and/or the hair to appear aesthetically pleasing. There is a great need to find active ingredients that can protect the skin, scalp and hair from the aggressions of the exposome, e.g., the aggressions of the environment to which they may be exposed to on a daily basis, including stressors due to daily personal care routines. In particular, there is a great need to find such active ingredients that can provide such protection to the aesthetic benefit of the skin and/or the hair.

US20210177733 discloses a composition and method for enhancing the skin's ability to defend itself against free radical aggression caused by exposome-induced oxidative stress. The compositions comprise leaf extract of Aristotelia chilensis; leaf extract of Buddleja globosa; and leaf extract of Ugni molinae.

Zoryalys® from Greentech is a natural active ingredient “derived from several molecules: Shogaol & Gingerol (grows in sunny areas), Magnolol & Honokiol (lives in polluted areas) and Pracaxi (biomimetic lipid)”. ZORYALYS® is described as protecting the hair against protein oxidation and being active from the surface to the heart of the hair fiber allowing a full protection of hair against exposome aggressions. This active is described as maintaining hair mechanical properties and improving hair shine for a healthy and beautiful hair, by maintaining keratin integrity.

Peptides released from extruded adzuki bean protein through simulated gastrointestinal digestion that exhibit anti-inflammatory activity are disclosed in Zhenxing Shi et al., “Peptides released from extruded adzuki bean protein through simulated gastrointestinal digestion exhibit anti-inflammatory activity”, J. Agric. Food Chem. 2021, 69, 7028-7036.

There remains a need for alternative plant-based active compositions that can protect the skin and/or hair against exposome-induced oxidative stress. There remains a need for alternative plant-based active compositions that can protect/maintain/improve the aesthetic properties of the skin and/or hair when exposed to the exposome.

The present invention sets out to meet some or all of the above-identified needs and to solve some or all of the above-identified problems.

### SUMMARY OF THE INVENTION

In a first aspect, the invention relates to a protein hydrolysate of azuki beans comprising at least one compound (i.e., 1, 2, 3 or 4 compounds) chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-GIn-Gly-His-Asp-Arg;
(iii)
(SEQ ID No: 3)
Val-Asp-Ala-Gln-Pro-Glu-Glu-Gln-Gln-Arg;
and
(iv)
(SEQ ID No: 4)
Gly-Ile-Asn-Ala-Glu-Asn-Asn-Gln-Arg-Asn.

The protein hydrolysate may additionally comprise the compound:

(v)
(SEQ ID No: 5)
Glu-Tyr-Thr-Gly-Asp-Ala-Ala-Gly-Arg.

It has been found that the protein hydrolysate of the invention is useful in alleviating or preventing adverse effects of the exposome on the skin and/or hair. Adverse effects to the skin and/or hair include those arising from air pollution and/or sunlight. Adverse effects to the hair also include those due to chemical treatment such as dyeing and physical treatment such as from styling aids, e.g., hair dryers or straightening irons. Adverse effects to hair can manifest themselves in the form of hair that: is damaged; is less manageable; has more split ends; has reduced fiber alignment; has increased frizz; and/or is less hydrated. Adverse effects to skin can manifest themselves in the form of a reduction in barrier function; a dull appearance of the skin; a loss of brightness, luminosity and/or glossiness of the skin; a loss in homogeneity in the texture of the skin; an increase in roughness of the skin; the appearance of dark spots; and/or erythema (skin redness).

In another aspect, the invention relates to a compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg,

and
stereoisomers, and cosmetically acceptable salts thereof. Surprisingly, it has been found that these compounds exhibit excellent antioxidant activity. Without wishing to be bound by theory, it is believed that this excellent antioxidant activity of these compounds contributes to the ability of the protein hydrolysates to perform as described herein.

In one aspect, the invention relates to combination of the protein hydrolysate according to the first aspect of the invention and a filtrate obtained by the fermentation of Raphanus sativus root by the microorganism Leuconostoc. It has been found that this combination results in a synergistic effect with respect to antioxidant activity. The invention also extends to the combination of a compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg,

and
stereoisomers and cosmetically acceptable salts thereof and a filtrate obtained by the fermentation of Raphanus sativus root by the microorganism Leuconostoc.

In one aspect, the invention relates a process for preparing a protein hydrolysate of azuki beans, wherein the hydrolysate is obtained by:
a) providing a suspension of ground azuki beans in water;
b) treating the suspension with alkali so as to solubilize proteins in the ground azuki beans;
c) enzymatically treating the suspension obtained in b) with a serine endoprotease;
d) deactivating the serine endoprotease in the suspension obtained in c) to form a crude hydrolysate;
e) recovering a supernatant from the crude hydrolysate by solid/liquid separation; and
f) removing components from the supernatant having a molecular weight of greater than 3 KDa.

The process is useful in preparing protein hydrolysates according to the first aspect of the invention. The invention extends to protein hydrolysates obtained by the process of preparing a protein hydrolysate of azuki beans described herein.

In one aspect the invention relates to a personal care or cosmetic composition comprising at least one cosmetically acceptable excipient or ingredient and a protein hydrolysate of azuki beans according to the first aspect of the invention, or a compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg,

and
stereoisomers, and cosmetically acceptable salts thereof, or a combination of said compound and a filtrate obtained by the fermentation of Raphanus sativus root by the microorganism Leuconostoc.

In one aspect, the invention relates to the use of a protein hydrolysate of azuki beans according to the first aspect of the invention, or a compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg,

and
stereoisomers, and cosmetically acceptable salts thereof, or a combination of said compound and a filtrate obtained by the fermentation of Raphanus sativus root by the microorganism Leuconostoc for the treatment and/or care of the skin and/or hair. In particular, the treatment and/or care of the skin and/or hair is cosmetic and non-therapeutic.

In one aspect, the invention relates to a method of treatment and/or care of the skin and/or hair comprising administering a protein hydrolysate of azuki beans according to the first aspect of the invention, or a compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg,

and
stereoisomers, and cosmetically acceptable salts thereof, or a combination of said compound and a filtrate obtained by the fermentation of Raphanus sativus root by the microorganism Leuconostoc to a subject. The treatment and/or care includes: the alleviation or prevention of the adverse effects of the exposome on the skin and/or hair; the alleviation or prevention of the adverse effects of air pollution on the skin and/or hair; the alleviation or prevention of the adverse effects of sunlight on the skin and/or hair; the alleviation or prevention of the adverse effects of chemical treatment of the hair; the alleviation or prevention of the adverse effects of heat stress on the hair; and/or the maintenance and/or improvement of the physical barrier function of the skin.

### DETAILED DESCRIPTION OF THE INVENTION

The invention is based on the finding of surprising properties of a protein hydrolysate of azuki beans, which properties lend the protein hydrolysate to cosmetic, non-therapeutic applications on the skin and/or hair.

### Definitions

In the context of this invention “skin” is understood to be the layers which comprise it, from the uppermost layer or stratum corneum to the lowermost layer or hypodermis, both inclusive. These layers are composed of different types of cells such as keratinocytes, fibroblasts, melanocytes and/or adipocytes among others. In the context of this invention, the term “skin” includes the scalp. The term “skin” includes the skin of mammals, for example, the skin of humans, and includes skin comprising hair.

In the context of this invention “hair” includes the hair of the scalp, the skin, the eyelashes, the eyebrows, moustache region and/or beard region of a subject.

The term “treatment” as used in the context of this specification when accompanied by the qualification “cosmetic”, it means that the treatment is non-therapeutic and has the aim of improving the aesthetic appearance of the skin and/or the hair and includes improving the properties of the skin and/or hair such as, but not restricted to, the level of hydration, elasticity, firmness, shine, tone or texture, which properties affect the cosmetic appearance of the skin and/or hair. The term “care” in the context of this specification refers to the maintenance of the properties of the skin and/or hair. The properties of the skin and/or hair are subject to improvement and maintenance through cosmetic treatment and/or care both in healthy subjects as well as those who present diseases and/or disorders of the skin and/or hair, such as and not restricted to, ulcers and lesions on the skin, psoriasis, dermatitis, acne or rosacea, alopecia, among others.

The term “prevention”, as used for this invention, refers to the ability of the active of the invention to prevent, delay or hinder the appearance or development of a symptom or manifestation of an adverse effect on the skin and/or hair. The term “alleviation”, as used for this invention refers to the ability of the active of the invention to reduce or lessen a symptom or manifestation of an adverse effect on the skin and/or hair.

The term “about” as used herein, e.g., when referring to a measurable value (such as an amount or weight of a particular component or temperature), refers to variations of ±20%, ±10%, ±5%, ±1%, ±0.5%, or, particularly, ±0.1% of the specified amount.

As used herein, the term “comprising”, which is inclusive or open-ended and does not exclude additional unrecited elements or method steps, is intended to encompass as alternative embodiments, the phrases “consisting essentially of” and “consisting of” where “consisting of” excludes any element or step not specified and “consisting essentially of” permits the inclusion of additional unrecited elements or steps that do not materially affect the essential or basic and novel characteristics of the composition or method under consideration.

In this description the abbreviations used for amino acids follow the rules of IUPAC-IUB Commission of Biochemical Nomenclature specified in Eur. J. Biochem., (1984), 138, 9-37. Thus, for example, Gly represents NH₂—CH₂—COOH, Gly-represents NH₂—CH₂—CO—, -Gly represents-NH—CH₂—COOH and -Gly-represents-NH—CH₂—CO—.

Therefore, the hyphen, which represents the peptide bond, eliminates the OH in the 1-carboxyl group of the amino acid (represented here in the conventional non-ionized form) when situated to the right of the symbol, and eliminates the H of the 2-amino group of the amino acid when situated to the left of the symbol; both modifications can be applied to the same symbol (see the table below).

Structures of the amino acid residues and their nomenclature in one and
three-letter code

| Name | Residue | Symbol | Residue |
|---|---|---|---|
| Asparagyl -Asn- N | 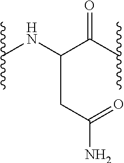 [SMILES: *NC(CC(N)=O)C(*)=O] | Glutaminyl -Gln- Q | 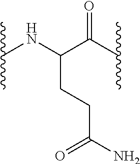 [SMILES: *NC(CCC(N)=O)C(*)=O] |
| Histidyl -His- H | 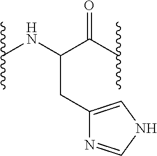 [SMILES: *NC(Cc1c[nH]cn1)C(*)=O] | Glycyl -Gly- G | 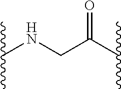 [SMILES: *NCC(*)=O] |
| Lysyl -Lys- K | 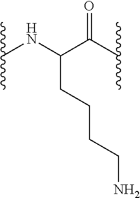 [SMILES: *NC(CCCCN)C(*)=O] | Tyrosyl -Tyr- Y | 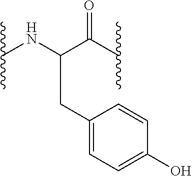 [SMILES: *NC(Cc1ccc(O)cc1)C(*)=O] |
| Leucyl -Leu- L | 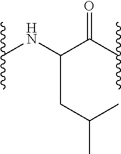 [SMILES: *NC(CC(C)C)C(*)=O] | Aspartyl -Asp- D | 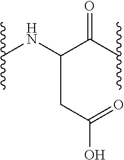 [SMILES: *NC(CC(=O)O)C(*)=O] |
| Glutamyl -Glu- E | 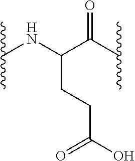 [SMILES: *NC(CCC(=O)O)C(*)=O] | Isoleucyl -Ile- I | 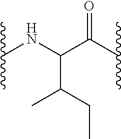 [SMILES: *NC(C(*)=O)C(C)CC] |
| Valyl -Val- V | 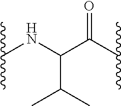 [SMILES: *NC(C(*)=O)C(C)C] | Arginyl -Arg- R | 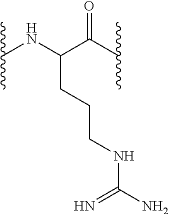 [SMILES: *NC(CCCNC(=N)N)C(*)=O] |
| Threonyl -Thr- T | 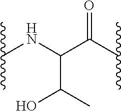 [SMILES: *NC(C(*)=O)C(C)O] | Prolyl -Pro- P | 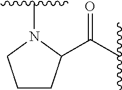 [SMILES: *C(=O)C1CCCN1*] |
| Alanyl -Ala- A | 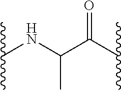 [SMILES: *NC(C)C(*)=O] | | |

### Protein Hydrolysate of Azuki Beans and Process for Preparing Same

In a first aspect, the invention relates to a protein hydrolysate of azuki beans.

The azuki bean is also known as Vigna angularis and is a legume that was originally grown in China and high-altitude terrains near Tibet, and is consumed worldwide because it is a rich source of proteins (percentage>20%). Vigna angularis is also known as adzuki bean (Japanese: [symbol] ), azuki, [symbol] , adzuki), azuki bean, or red mung bean.

A protein hydrolysate is a mixture of fragments obtained from the hydrolytic degradation of proteins and comprises, e.g., free amino acids and peptides of different molecular weights and compositions. The protein hydrolysate of the invention can be obtained from the enzymatic hydrolysis of proteins of azuki beans.

The protein hydrolysate comprises at least one compound (i.e., 1, 2, 3 or 4 compounds) chosen from:

(i)
(SEQ ID No. 1)
Val-Ala-Glu-Gln-Glu-Gln-Glu-Gln-Ala-Lys-Arg-Gln;
(ii)
(SEQ ID No. 2)
Leu-Pro-Gln-Gln-Glu-Gln-Gly-His-Asp-Arg;
(iii)
(SEQ ID No. 3)
Val-Asp-Ala-Gln-Pro-Glu-Glu-Gln-Gln-Arg;
and
(iv)
(SEQ ID No. 4)
Gly-Ile-Asn-Ala-Glu-Asn-Asn-Gln-Arg-Asn.

Thus, the protein hydrolysate can comprise:

(i)
(SEQ ID No. 1)
Val-Ala-Glu-Gln-Glu-Gln-Glu-Gln-Ala-Lys-Arg-Gln;
(ii)
(SEQ ID No. 2)
Leu-Pro-Gln-Gln-Glu-Gln-Gly-His-Asp-Arg.

The protein hydrolysate can comprise:

(iii)
(SEQ ID No. 3)
Val-Asp-Ala-Gln-Pro-Glu-Glu-Gln-Gln-Arg;
and
(iv)
(SEQ ID No. 4)
Gly-Ile-Asn-Ala-Glu-Asn-Asn-Gln-Arg-Asn.

The protein hydrolysate can comprise:

(i)
(SEQ ID No. 1)
Val-Ala-Glu-Gln-Glu-Gln-Glu-Gln-Ala-Lys-Arg-Gln;
(iii)
(SEQ ID No. 3)
Val-Asp-Ala-Gln-Pro-Glu-Glu-Gln-Gln-Arg;
and
(iv)
(SEQ ID No. 4)
Gly-Ile-Asn-Ala-Glu-Asn-Asn-Gln-Arg-Asn.

The protein hydrolysate can comprise:

(ii)
(SEQ ID No. 2)
Leu-Pro-Gln-Gln-Glu-Gln-Gly-His-Asp-Arg;
(iii)
(SEQ ID No. 3)
Val-Asp-Ala-Gln-Pro-Glu-Glu-Gln-Gln-Arg;
and
(iv)
(SEQ ID No. 4)
Gly-Ile-Asn-Ala-Glu-Asn-Asn-Gln-Arg-Asn.

The protein hydrolysate can comprise:

(i)
(SEQ ID No. 1)
Val-Ala-Glu-Gln-Glu-Gln-Glu-Gln-Ala-Lys-Arg-Gln;
(ii)
(SEQ ID No. 2)
Leu-Pro-Gln-Gln-Glu-Gln-Gly-His-Asp-Arg;
(iii)
(SEQ ID No. 3)
Val-Asp-Ala-Gln-Pro-Glu-Glu-Gln-Gln-Arg;
and
(iv)
(SEQ ID No. 4)
Gly-Ile-Asn-Ala-Glu-Asn-Asn-Gln-Arg-Asn.

The protein hydrolysate, in particular the protein hydrolysate having any of the combinations of compounds labelled (i), (ii), (iii) or (iv) given above, may additionally comprise the compound:

(v)
(SEQ ID No. 5)
Glu-Tyr-Thr-Gly-Asp-Ala-Ala-Gly-Arg.

Each of the compounds labelled (i) to (v) above has been found to have excellent antioxidant activity. Without wishing to be bound by theory, it is believed that this activity contributes to the advantageous skin/hair treatment and/or care properties exhibited by the protein hydrolysate of the invention.

When present in the protein hydrolysate, each of these compounds labelled (i) to (v) above is present in the protein hydrolysate in an amount of from 0.001 to 0.15 ppm or 0.005 to 0.08 ppm. When present in the protein hydrolysate, the compound labelled (i) above can be present in the protein hydrolysate in an amount of from 0.001 to 0.1 ppm, 0.005 to 0.05 ppm, or 0.01 to 0.03 ppm. When present in the protein hydrolysate, the compound labelled (ii) above can be present in the protein hydrolysate in an amount of from 0.001 to 0.1 ppm, 0.005 to 0.05 ppm, or 0.015 to 0.04 ppm. When present in the protein hydrolysate, the compound labelled (iii) above can be present in the protein hydrolysate in an amount of from 0.001 to 0.1 ppm, 0.005 to 0.05 ppm, or 0.01 to 0.03 ppm. When present in the protein hydrolysate, the compound labelled (iv) above can be present in the protein hydrolysate in an amount of from 0.005 to 0.15 ppm, 0.01 to 0.1 ppm, or 0.03 to 0.08 ppm. When present in the protein hydrolysate, the compound labelled (v) above can be present in the protein hydrolysate in an amount of from 0.001 to 0.15 ppm, 0.001 to 0.01 ppm, or 0.003 to 0.008 ppm.

The protein hydrolysate can have a molecular weight of less than or equal to 3 kDa. Thus, the protein hydrolysate can be absent of any components having a molecular weight of greater than 3 kDa, i.e., it can contain no components having a molecular weight of greater than 3 kDa. This can be achieved by filtering the hydrolysate.

The protein hydrolysate can be obtained by treating a suspension of ground azuki beans in water with alkali to solubilize proteins in the ground azuki beans, treating the resultant suspension with a serine endoprotease, deactivating the serine endoprotease in the suspension to form a crude hydrolysate, and purifying the crude hydrolysate. In this case, the enzymatic hydrolysis of the proteins in the ground azuki beans is carried out in a suspension (a suspension of ground azuki beans in water in which proteins in the azuki beans have been solubilized), not on any form of isolated protein. Purifying can comprise removing any components having a molecular weight of greater than 3 kDa from the crude hydrolysate. The protein hydrolysate of the invention is the purified product of this process. The process may not comprise steps of isolating proteins from the ground azuki beans and subjecting the isolated proteins to enzymatic hydrolysis. The process may not comprise subjecting proteins isolated from azuki beans to enzymatic hydrolysis and, as a result the protein hydrolysate will comprise components other than fragments of proteins, such as polyphenols and other organic compounds.

In particular, the protein hydrolysate can comprise polyphenols in an amount of at least 0.05 mg of polyphenols per milliliter of protein hydrolysate, particularly at least 0.15 mg of polyphenols per milliliter of protein hydrolysate. In particular the amount of polyphenols can be in the range of 0.05 to 0.30 mg/mL, more particularly 0.15 to 0.25 mg/mL. The amount of polyphenols may be measured by the Folin-Ciocalteu method and can particularly expressed as mg of gallic acid equivalents per mL of protein hydrolysate. The Folin-Ciocalteu method is a well-known method for a person skilled in the analysis of compositions derived for botanicals and is extensively described in the literature (e.g., Lamuela-Raventós, “Folin-Ciocalteu method for the measurement of total phenolic content and antioxidant capacity” 2018, Measurement of Antioxidant Activity & Capacity: Recent Trends and Applications). Alternatively, the protein hydrolysate can comprise polyphenols in an amount of at least 2 mg of polyphenols per g of protein hydrolysate (in dry weight), particularly at least 6 mg/g. In particular the amount of polyphenols can be in the range 2 to 12 mg/g, more particularly 6 to 9 mg/g. By “polyphenols” is meant naturally occurring organic compounds characterized by the presence of multiple phenol units. Polyphenols include for example flavonoids, tannic acid, and ellagitannin.

The protein hydrolysate can have a dry weight of 2 to 5 mg; a protein content of 19 to 35 w/w %; a DPPH activity of 74 to 80%; and a polyphenols content of 0.05 to 0.30 mg/mL or 2 to 12 mg/g.

The protein hydrolysate can be prepared according to the following steps: a) to d) to obtain a crude hydrolysate, followed by purification of the crude hydrolysate.

Step a) involves providing a suspension of ground azuki beans in water. The azuki beans are raw azuki beans. The ground azuki beans can be obtained by grinding azuki beans into a flour. The flour can be sifted to remove larger fragments. Typically, the ground azuki beans have a particle size of less than 250 micrometers.

Step b) involves treating the suspension of ground azuki beans in water with alkali so as to solubilize proteins of the ground azuki beans in the water. The purpose of this step is to increase the content of protein solubilized in the suspension. Typically, this step involves adjusting the pH of the suspension to a pH of from about 6.5 to about 7.5, for example, to a pH of 7. The pH of the suspension affects the amount and type of proteins in the suspension. This can be achieved by adding an alkali such as NaOH to the suspension of ground azuki beans in water. Typically step b) is carried out at room temperature, i.e., at 22±2° C. After the alkali has been added to the suspension of ground azuki beans, the suspension is maintained at room temperature for a period of time, typically 1 hour, prior to step c).

Step c) involves enzymatically treating the suspension obtained in step b) with a serine endoprotease (also known as a serine endopeptidase). The serine endoprotease can be a subtilisin A. Subtilisin A (classification EC 3.4. 21.62, peptidase family S8) is also known as Subtilisin Carlsberg, Subtilopeptidase A, Alcalase®, ALK-enzyme, bacillopeptidase A, Bacillus subtilis alkaline proteinase bioprase, bioprase AL, colistinase, genenasel, Esperase®, maxatase, protease XXVII, thermoase, superase, subtilisin DY, subtilopeptidase, SP 266, Savinase®, kazusase, protease VIII, protin A 3 L, orientase 10B, or protease S. Subtilisin A is commonly obtained from Bacillus licheniformis. Step c) is carried out at a temperature of from 20 to 30° C., and can be carried out at a temperature of from 23 to 27° C. or at a temperature of about 25° C. Step c) can be carried out for a period of about 30 to about 120, about 45 to about 90, or about 60 minutes, for example. Typically, the enzyme activity per g of protein in the ground azuki beans is from 0.1 to 0.5 AU/g. AU represents Anson Units. An Anson Unit is a unit of enzyme concentration which is defined as the amount of enzyme that can digest urea-denatured hemoglobin at the same initial rate as one milliequivalent of tyrosine at standard conditions (25 C and pH 7.50). Typically, the percentage of protein in the ground azuki beans is from 15 to 25% w/w. Typically, the concentration of ground azuki beans in water is 20-100 g (ground azuki beans)/Kg (water).

Step d) involves deactivating the enzyme to give a crude isolate. During step c) the pH of the suspension (undergoing enzymatic hydrolysis) is monitored. A drop of pH is associated with the release of peptide fragments and subsequent buildup of acidic carboxylic acids. Typically, during step c) the suspension undergoing enzymatic hydrolysis suffers slight acidification, resulting in a drop of 0.2 to 0.3 pH units, for example. After the enzymatic hydrolysis step c), the enzyme is deactivated by increasing the temperature of the suspension obtained in c) to at least 70° C., or to from 70 to 90° C., or to from 75 to 85° C. The suspension obtained in step c) can be obtained at this temperature for a period of time, preferably for at least about 20 minutes. The product of step d) is crude hydrolysate.

The crude hydrolysate product of step d) is then subjected to purification to remove any components having a molecular weight of greater than 3 kDa. This can be achieved by solid/liquid separation (step e)) and ultrafiltration (step f)). Step e) is a purification step and involves removing solid material from the crude hydrolysate by solid/liquid separation. Suitable separation techniques are known in the art and include, for example, filtration, sedimentation, decantation and/or centrifugation. Filtration can be carried out using filters having a pore size of lower than 1000 μm, or lower than 20 μm, or lower than 10 μm, or lower than 1 μm, or lower than 0.1 μm. Filtration may be performed in successive filtration operations, for instance using filters of decreasing pore size. Step f) is also a purification step and involves ultrafiltration to remove fragments from the supernatant obtained in step e) that have a molecular weight of greater than 3 kDa. Ultrafiltration is a technique that is well known in the art and involves the use of a membrane to separate molecules with different molecular weights, in this case with a cut off threshold of 3 kDa.

The invention extends to a protein hydrolysate obtained by the above described process.

The invention extends to a compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
and
(v)
(SEQ ID No: 5)
Glu-Tyr-Thr-Gly-Asp-Ala-Ala-Gly-Arg.

Each of the compounds labelled (i) to (v) above has been found to have excellent antioxidant activity. Without wishing to be bound by theory, it is believed that this activity contributes to the advantageous skin/hair treatment and/or care properties exhibited by the protein hydrolysate of the invention.

The invention also extends to a combination of the protein hydrolysate according to the first aspect of the invention and a filtrate obtained by the fermentation of Raphanus sativus root by the microorganism Leuconostoc.

The invention extends to the combination of a compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg;

and
stereoisomers and cosmetically acceptable salts thereof and a filtrate obtained by the fermentation of Raphanus sativus root by the microorganism Leuconostoc.

This filtrate obtained by the fermentation of Raphanus sativus (radish) root by the microorganism Leuconostoc contains antimicrobial peptides and acts as a preservative. One example of a commercial filtrate obtained by the fermentation of Raphanus sativus root by the microorganism Leuconostoc is Leucidal® (INCI Name: Leuconostoc/Radish Root Ferment Filtrate) from Active Micro Technologies LCC. The most preferable species of Leuconostoc is Leuconostoc kimchi. Leucidal® is typically employed as a preservative. It has been found that the antioxidant activity of the protein hydrolysate of the invention is unexpectedly boosted in the presence of this filtrate.

### Compositions

The invention includes a composition which may be a personal care composition or a cosmetic composition, and which comprises the protein hydrolysate as described herein, together with at least one cosmetically acceptable excipient or adjuvant. The invention extends to a composition which may be a personal care composition or a cosmetic composition and which comprises: a combination of the protein hydrolysate as described herein and a filtrate obtained by the fermentation of Raphanus sativus root by the microorganism Leuconostoc; together with at least one cosmetically acceptable excipient or adjuvant. The invention extends to a composition which may be a personal care composition or a cosmetic composition, comprising a compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg,

and
stereoisomers, and cosmetically acceptable salts thereof, together with at least one cosmetically acceptable excipient or adjuvant. The invention extends to a composition which may be a personal care composition or a cosmetic composition and which comprises: a combination of a compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg,

and
stereoisomers, and cosmetically acceptable salts thereof, and a filtrate obtained by the fermentation of Raphanus sativus root by the microorganism Leuconostoc; together with at least one cosmetically acceptable excipient or adjuvant.

These compositions can be prepared by conventional means known to persons skilled in the art [“Harry's Cosmeticology”, Seventh edition, (1982), Wilkinson J. B., Moore R. J., ed. Longman House, Essex, GB].

The compositions contain a cosmetically effective amount of the protein hydrolysate, or compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg,

and
stereoisomers, and cosmetically acceptable salts thereof, which should be administered, as well as their dosage, will depend on numerous factors, including age, state of the patient, the nature or severity of the condition, disorder or disease to be treated and/or cared for, the route and frequency of administration and of the particular nature of the compounds to be used.

The terms “cosmetically effective amount” is understood to mean a non-toxic but sufficient amount of extract of the invention to provide the desired effect. The protein hydrolysate can be present in an amount of at least 0.001 wt %, particularly from about 0.001 to 10 wt %, preferably from about 0.004 to 5 wt %, based on the total weight of the personal care or cosmetic composition.

The compositions can be compositions for topical application which optionally include cosmetically acceptable excipients necessary for formulating the desired administration form. Topical compositions are compositions suitable for the topical application to mammalian keratinous tissue such as skin or hair.

Compositions for topical or transdermal application can be produced in any solid, liquid or semisolid formulation, such as and not restricted to, creams, multiple emulsions such as and not restricted to, oil and/or silicone in water emulsions, water-in-oil and/or silicone emulsions, water/oil/water or water/silicone/water type emulsions and oil/water/oil or silicone/water/silicone type emulsions, anhydrous compositions, aqueous dispersions, oils, milks, balsams, foams, lotions, gels, cream gels, hydroalcoholic solutions, hydroglycolic solutions, hydrogels, liniments, sera, soaps, shampoos, conditioners, serums, polysaccharide films, ointments, mousses, pomades, powders, bars, pencils and sprays or aerosols (sprays), including leave-on and rinse-off formulations. These topical or transdermal application formulations can be incorporated using techniques known by the person skilled in the art into different types of solid accessories for example and not restricted to, bandages, gauzes, t-shirts, socks, tights, underwear, girdles, gloves, diapers, sanitary napkins, dressings, bedspreads, wipes, adhesive patches, non-adhesive patches, occlusive patches, microelectric patches or face masks, or they can be incorporated into different make-up products such as make-up foundation, such as fluid foundations and compact foundations, make-up removal lotions, make-up removal milks, under-eye concealers, eye shadows, lipsticks, lip protectors, lip gloss and powders among others.

The compositions of the invention can be in a form chosen from creams, multiple emulsions, solutions, liquid crystals, anhydrous compositions, aqueous dispersions, oils, milks, balsams, foams, lotions, gels, cream gels, hydroalcoholic solutions, hydroglycolic solutions, hydrogels, liniments, soaps, shampoos, conditioners, serums, polysaccharide films, ointments, mousses, pomades, powders, bars, pencils, sprays or aerosols.

The compositions of the invention may include agents which increase the percutaneous absorption of the compounds of the invention, for example and not restricted to, dimethylsulfoxide, dimethylacetamide, dimethylformamide, surfactants, azone (1-dodecylazacycloheptane-2-one), alcohol, urea, ethoxydiglycol, acetone, propylene glycol or polyethylene glycol, among others.

In particular, the compositions are for topical application and are hair care compositions such as conditioners, treatments, hair tonics, styling gels, mousses, shampoos, hair sprays, pomades, setting lotions, coloring and permanent waving compositions. Of particular interest for the purpose of the present invention are tonics, conditioners, treatments, and styling gels which may be in the form of a gel, a lotion, a tincture, a spray, a mousse, a cleansing composition or a foam and which may be applied according to individual needs, e.g., once daily as a lotion, tincture, mousse or spray; or once or twice weekly as a conditioner or treatment. Particularly suitable compositions in the context of this invention are lotions, a shampoo, a serum or eyelashes mascara. Particularly, the cosmetic composition is a serum. The term “serum” is well known by the person skilled in the art and refers to compositions that are clear, gel-based or liquid. Serum has a fluid texture and is more concentrated in active agents than a standard care product.

Further, the compositions may include other active ingredients such as anti-wrinkle agents, botox-like agents and/or anti-aging agents; firming agents, skin elasticity agents and/or restructuring agents; moisturizing agents; anti-photoaging agents, and/or blue-light protector agents; DNA protecting agents, DNA repair agents, and/or stem cell protecting agents; free radical scavengers and/or anti-glycation agents, detoxifying agents, antioxidant and/or anti-pollution agents; anti-perspirant agents; melanin synthesis stimulating or inhibiting agents; whitening or depigmenting agents; propigmenting agents; self-tanning agents; lipolytic agents or agents stimulating lipolysis, adipogenic agents, etc. Additional examples can be found in CTFA International Cosmetic Ingredient Dictionary & Handbook, 12th Edition (2008).

The anti-wrinkle agent, botox-like agent and/or anti-aging agent can be Oxygestkin® [INCI: Tropaeolum majus Flower/Leaf/Stem Extract] or Eclaline™. [INCI: Hydrolyzed Lupine Protein Octenylsuccinate] from SILAB; Matrixyl® [INCI: Palmitoyl Pentapeptide-4], Matrixyl® 3000® [INCI: Palmitoyl Tetrapeptide-7, Palmitoyl Oligopeptide], Matrixyl® Synthe'6 [INCI: Glycerin, Water, Hydroxypropyl Cyclodextrin, Palmitoyl Tripeptide-38], Matrixyl® Morphomics™ [INCI: Pentylene Glycol, Caprylyl Glycol], Essenskin™ [INCI: calcium hydroxymethionine], Renovage™ [INCI: Teprenone], Dermaxyl® [INCI: Palmitoyl Oligopeptide], Calmosensine™ [INCI: Butylene Glycol, Acetyl Dipeptide-1 Cetyl Ester], Volulip™ [INCI: Cetearyl Ethylhexanoate, Sorbitan Isostearate, Portulaca Pilosa Extract, Sucrose Cocoate, Palmitoyl Tripeptide-38], Subliskin™ [INCI: Sinorhizobium Meliloti Ferment, Cetyl Hydroxyethyl Cellulose, Lecithin], Biopeptide CL [INCI: Palmitoyl Oligopeptide], Biopeptide EL [INCI: Palmitoyl Oligopeptide], Rigin™ [INCI: Palmitoyl Tetrapeptide-3], Biobustyl™ [INCI: Glyceryl Polymethacrylate, Rahnella/Soy Protein Ferment, Palmitoyl Oligopeptide], Dynalift™ [INCI: Sodium Polystyrene Sulfonate, Sorghum Bicolor Stalk Juice, Glycerin], Idealift™ [INCI: Acetyl Dipeptide-1 Cetyl Ester], Siegesbeckia [INCI: Siegesbeckia Orientalis Extract], Ovaliss™ [INCI: Coco-glucoside, Caprylyl Glycol, Alcohol, Glaucine], Juvinity™ [INCI: Geranylgeranyl isopropanol], Prolevis™ [INCI: Hydrolyzed Vegetable Protein], Idealift™ [INCI: Hydroxyethyl cellulose, Acetyl Dipeptide-1 cetyl ester], Beautifeye™ [INCI: Albizia Julibrissin Bark Extract, Darutoside], Chromocare™ [INCI: Sigesbeckia Orientalis Extract, Rabdosia rubescens Extract] or Resistem™ [INCI proposed: Globularia Cordifolia Ferment] marketed by Sederma/Croda. Vialox® [INCI: Pentapeptide-3], Syn®-Ake® [INCI: Dipeptide Diaminobutyroyl Benzylamide Diacetate], Syn®-Coll [INCI: Palmitoyl Tripeptide-5], Phytaluronate™ [INCI: Ceratonia Siliqua (Carob) Gum], Preregen® [INCI: Glycine soja (Soybean) Protein, Oxidoreductases], Pepha-Nutrix™ [INCI: Natural Nutrition Factors], Pepha-Tight™ [INCI: Algae Extract, Pullulan], Pentacare-NA™ [INCI: Hydrolyzed Wheat Gluten, Ceratonia Siliqua Gum], Syn®-Tacks [INCI: Glycerin, Palmitoyl Dipeptide-5 Diaminobutyroyl Hydroxythreonine, Palmitoyl Dipeptide-6 Diamino hydroxybutyrate], BeauActive™ MTP [INCI: Hydrolyzed milk protein], Syn®-TC [INCI: Tetradecyl Aminobutyroylvalylaminobutyric Urea Trifluoroacetate, Palmitoyl Tripeptide-5, Palmitoyl Dipeptide-5 Diaminobutyroyl Hydroxythreonine], Syn®-Hycan [INCI: Tetradecyl Aminobutyroylvalylaminobutyric Urea Trifluoroacetate], Syn®-Glycan [INCI: Tetradecyl Aminobutyroylvalyl-aminobutyric Urea Trifluoroacetate], Regu-Age™ [INCI: Hydrolyzed Rice Bran Protein, Oxidoreductases, Glycine Soja Protein], Pepha-Timp™ [INCI: Human oligopeptide-20], Pepha-Age [INCI: Dunaliella Salina Extract], Colhibin™ [INCI: Hydrolyzed Rice Protein], Elhibin™ [INCI: Glycine Soja Protein, Disodium cocoamphodiacetate] or All-Q™ Plus [INCI: Ubiquinone, Tocopheryl Acetate] marketed by Pentapharm/DSM; Myoxinol™ [INCI: Hydrolyzed Hibiscus esculentus Extract], Myoxinol™ LS 9736 [INCI: Hydrolyzed Hibiscus esculentus Extract, Dextrin], Syniorage™ [INCI: Acetyl Tetrapeptide-11], Dermican™ [INCI: Acetyl Tetrapeptide-9], DN-AGE® LS [INCI: Cassia alata leaf Extract], Hyalufix™ GL [INCI: Alpinia Galanga Leaf Extract], Neurobiox™ [INCI: Achillea Millefolium Extract], Deliner™ [INCI: Zea Mays (Corn) Kernel Extract], Lys'lastine™ V [INCI: Peucedanum Graveolens (Dill) Extract], Extracellium™ [INCI: Hydrolyzed Potato Protein], Proteasyl™ TP LS 8657 [INCI: Pisum sativum Extract], Flavagrum™ PEG [INCI: PEG-6 Isostearate, Hesperetin Laurate], Micromerol™ [INCI: Pyrus malus Fruit Extract], Extracellium™ [INCI: Hydrolyzed Potato Protein], Marine Filling Spheres [INCI: Pentaerythrityl Tetraisostearate, Silica Dimethyl Silylate, Sodium Chondroitin Sulfate, Atelocollagen], Triactigen™ [INCI: Mannitol, Cyclodextrin, Yeast Extract, Disodium Succinate], Eterniskin™ [INCI: Grifola Frondosa Fruiting Body Extract, Maltodextrin], Ascotide™ [INCI: Ascorbyl Phosphate Succinoyl Pentapeptide-12], Hyalurosmooth™ [INCI: Cassia Angustifolia Seed Polysaccharide], Indinyl™ CA [INCI: Cassia Angustifolia Seed Polysaccharide], Arganyl™ [INCI: Argania spinosa Leaf Extract], Sphingoceryl™ Veg [INCI: Phyto-ceramides], Vit-A-Like [INCI: Vigna Aconitifolia Seed Extract], Peptiskin™ [INCI: Arginine/Lysine polypeptide], Prodejine™ [INCI: Mannitol, Cyclodextrin, Yeast Extract, Disodium Succinate], Aqu'activ™ [INCI: Behenyl Alcohol, Glyceryl Oleate, Cocamide MIPA, Calcium Citrate], Elestan™ [INCI: Glycerin, Manilkara Leaf Extract], Hibiscin™ HP [INCI: Hibiscus esculentus Seed Extract], Collalift®18 [INCI: Khaya Senegalensis Bark], Collrepair™ DG [INCI: Hexylene Glycol, Niacin] or Litchiderm™ [INCI: Litchi Chinensis Extract] Pericarp marketed by Laboratoires Sérobiologiques/Cognis/BASF; Argireline® [INCI: Acetyl Hexapeptide-8], SNAP-7 [INCI: Acetyl Heptapeptide-4], SNAP-8 [INCI: Acetyl Octapeptide-3], Leuphasyl® [INCI: Pentapeptide-18], Inyline® [INCI: Acetyl Hexapeptide-30], Aldenine® [INCI: Hydrolyzed Wheat Protein, Hydrolyzed Soy Protein, Tripeptide-1], Preventhelia® [INCI: Diaminopropionoyl Tripeptide-33], Decorinyl® [INCI: Tripeptide-10 Citrulline], Decorinol® [INCI: Tripeptide-9 Citrulline], Trylagen® [INCI: Pseudoalteromonas Ferment Extract, Hydrolyzed Wheat Protein, Hydrolyzed Soy Protein, Tripeptide-10 Citrulline, Tripeptide-1], Eyeseryl® [INCI: Acetyl Tetrapeptide-5], Peptide AC29 [INCI: Acetyl Tripeptide-30 Citrulline], Relistase® [INCI: Acetylarginyltryptophyl Diphenylglycine], Thermostressine® [INCI: Acetyl Tetrapeptide-22], Lipochroman™ [INCI: Dimethylmethoxy Chromanol], Chromabright® [INCI: Dimethylmethoxy Chromanyl Palmitate], Antarcticine® [INCI: Pseudoalteromonas Ferment Extract], dGlyage® [INCI: Lysine HCl, Lecithin, Tripeptide-9 Citrulline], Vilastene™ [INCI: Lysine HCl, Lecithin, Tripeptide-10 Citrulline], Hyadisine® [INCI: Pseudoalteromonas Ferment Extract], Hyanify™ [INCI: Saccharide Isomerate], Diffuporine® [INCI: Acetyl Hexapeptide-37], Silusyne® [INCI: Soybean (Glycine Soja) Oil, Sorbitan Sesquioleate, Isohexadecane, Sodium Hyaluronate, Lauryldimonium Hydroxypropyl Hydrolyzed Soy Protein, Acetyl Hexapeptide-39], Adifyline® [INCI: Acetyl Hexapeptide-38], Delisens™ [INCI: Acetyl Hexapeptide-46], Telangyn™ [INCI: Acetyl Tetrapeptide-40], Reproage™ peptide [INCI: Acetyl Hexapeptide-8], Cellynkage™ marine ingredient [INCI: Saccharide Isomerate], Eyedeline™ marine ingredient [INCI: Plankton Extract], Uplevity™ [INCI: Acetyl Tetrapeptide-2], Seacode™ marine ingredient [INCI: Pseudoalteromonas Ferment Extract] or Serilesine® peptide solution [INCI: Hexapeptide-10]; Argireline® Amplified [INCI: Acetyl Hexapeptide-8], Dawnergy™ [INCI: Nonapeptide-1] marketed by Lipotec/Lubrizol; Sirtalice™ [INCI: Bacillus Ferment], Epitensive™ [INCI: Nicotiana Benthamiana Hexapeptide-40 SH-Oligopeptide-1], Scelleye™ [INCI: Nicotiana Benthamiana SH-Oligopeptide-2], Seadermium™ [INCI: Aqua, Glycerin, Bacillus Ferment], Pauseile™ [INCI: Aqua, Glycerin, Bacillus Ferment] or Neoclair™ pro [INCI: Aqua, Glycerin, Caprylyl Glycol, Acetyl Tetrapeptide-2] marketed by Lipotrue; Collaxyl® IS [INCI: Hexapeptide-9], Laminixyl IST [INCI: Heptapeptide], Orsirtine™ GL [INCI: Oryza sativa (Rice) Extract], D′Orientine™ IS [INCI: Phoenix dactylifera (Date) Seed Extract], Phytoquintescine™ [INCI: Einkorn (Triticum monococcum) Extract], Quintescine™ IS [INCI: Dipeptide-4], Peptide Vinci 01 [INCI: Penta-decapeptide-1], Peptide Vinci 02™ [INCI: Hexapeptide-3], Aquarize IST [INCI: Hydrolyzed Rice Extract], Lanablue™ [INCI: Algae extract], Ederline™ [INCI: Pyrus malus (Apple) Seed Extract], Dynachondrine™ ISR [INCI: Hydrolyzed Soy Protein], Prolixir S20™ [INCI: Dimer Tripeptide-43], Phytocohesine™ PSP [INCI: Sodium Beta-Sitosteryl Sulfate, Beta-Sitosterol], Perenityl™ IS [INCI: Pyrus Communis (Pear) Seed Extract], Caspaline 14™ [INCI: Hexapeptide-42], Peptide Q10™ [INCI: Pentapeptide-34 Trifluoroacetate], Survixyl IS™ [INCI: Pentapeptide-31], ChroNOgen™ [INCI: Tetrapeptide-26], Elixiance™ [INCI: Schinus Molle Extract], Harmoniance™ [INCI: Nelumbo nucifera Flower Extract], Serenityl™ [INCI: Marsdenia Condurango Bark Extract], Natriance™ Wrinkle-less [INCI: Hydrolyzed Corn Protein], Phytoneomatrix™ [INCI: Hydrolyzed Soybean Extract], Prolixir™ ICE [INCI: Hydrolyzed Rice Protein], PhytoRNx Baobab™ [INCI: Hydrolyzed Adansonia Digitata Extract], Natriance™ Renovate Extract [INCI: Hydrolyzed Linseed Extract], Natriance™ Self-Hydrate Extract [INCI: Pisum sativum Extract], Actopontine™ YST [INCI: Hydrolyzed Yeast Protein] or Telosense™ [proposed INCI: Hydrolyzed Soy Protein, Hydrolyzed Yeast Protein] marketed by Vincience/ISP/Ashland; BONT-L-Peptide™ [INCI: Palmitoyl Hexapeptide-19], TIMP Peptide [INCI: Acetylhexapeptide-20], ECM Moduline™ [INCI: Palmitoyl Tripeptide-28], Renaissance™ [INCI: Hydrolyzed Wheat Protein, Palmitoyl Decapeptide-21, Decapeptide-22, Oligopeptide-78, Zinc Palmitoyl Nonapeptide-14] or X50 Antiaging [INCI: Lactic Acid/glycolic Acid Copolymer, Polyvinyl Alcohol, Copper Palmitoyl Heptapeptide-14, Heptapeptide-15 Palmitate] marketed by Infinitec Activos; EquiStat™ [INCI: Pyrus malus Fruit Extract, Glycine soja Seed Extract], Juvenesce™ [INCI: Ethoxydiglycol and Caprylic Triglyceride, Retinol, Ursolic Acid, Phytonadione, Ilomastat], Ursolisome™ [INCI: Lecithin, Ursolic Acid, Atelocollagen, Xanthan Gum, Sodium chondroitin sulfate], Basaline™ [INCI: Hydrolyzed Malt Extract], Phytokine™ [INCI: Hydrolyzed Soy Protein], marketed by Coletica/Engelhard/BASF; Ameliox™ [INCI: Carnosine, Tocopherol, Silybum marianum Fruit Extract] or PhytoCellTec™ Malus domestica [INCI: Malus domestica Fruit Cell Culture], Lipobelle Soyaglicane™ [INCI: Soy Isoflavones], RoyalEpigen™ P5 [INCI: Butyrospermum parkii Butter, Hydrogenated Lecithin, Maltodextrin, Pentapeptide-48, Phenethyl Alcohol, Ethylhexylglycerin, Glycerin, Aqua] or DermCom™ [INCI: Crocus Chrysanthus Bulb Extract, Acacia Senegal Gum, Aqua/Water] marketed by Mibelle Biochemistry; ActiMatrix™ [INCI: Peptide based mushroom Extract], Peptamide™ 6 [INCI: Hexapeptide-11] marketed by Active Organics/Arch; and combinations thereof.

The firming agent, skin elasticity agent and/or restructuring agent can be selected from the group consisting of Argassential™ [INCI: C10-16 Alkyl Glucoside, Dicaprylyl Ether, Glycerin] or Replexium™ BC [INCI: Dimethyl Isosorbide, Polysorbate 20, Aqua, Acetyl Tetrapeptide-11, Acetyl Tetrapeptide-9] marketed by BASF; Prolevis™ [INCI: Hydrolyzed Vegetable Protein] or Poretect™ [INCI: Caprylic/capric Triglyceride, Sorbitan Trioleate, Apium graveolens Seed Extract, Linum usitatissimum Seed Extract] marketed by Sederma/Croda; Actifirm™ Ultra Advanced botanical ingredient [INCI: Centella Asiatica Extract, Rosmarinus officinalis Leaf Extract, Dipropylene Glycol, Alcohol, Echinacea angustifolia Leaf Extract] or Actifcol™ Advanced botanical ingredient [INCI: Aqua, Glycerin, Sodium Citrate, Lentinus Edodes Extract, Potassium Sorbate, Sodium Benzoate, Phytic Acid] marketed by Lipotec/Lubrizol; Densorphin™ [INCI: Vitex agnus Castus Extract, Aqua, Maltodextrin] or PhytoCellTec™ Nunatak® [INCI: Isomalt, Aqua, Saponaria pumila callus Culture Extract, Lecithin] marketed by Mibelle; and combinations thereof.

The moisturizing agent can be selected from the group consisting of AquaShuttle™ [INCI: Sorbitol, Laminaria digitata Extract, Diatomaceous Earth] marketed by Infinitec; Aqua-Osmoline™ [INCI: Ceratonia siliqua (Carob) Seed Extract] marketed by Vincience/ISP/Ashland; Hydralphatine™ Asia [INCI: Hydrogenated Starch Hydrolysate, Panthenol, Bambusa vulgaris Shoot Extract, Nelumbo nucifera Flower Extract, Nymphaea alba Root Extract] or Hydraporine™ [INCI: Betaine, Hydrogenated Lecithin, Honey, Pectin] marketed by Lucas Meyer Cosmetics/Unipex; PatcH2O™ [INCI: Trehalose, Urea, Serine, Glyceryl Polyacrylate, Algin, Sodium Hyaluronate, Pullulan], Aqu'activ™ [INCI: Behenyl Alcohol, Glyceryl Oleate, Cocamide MIPA], Irwinol® [INCI: Octyldodecanol, Irvingia gabonensis Kernel Butter, Hydrogenated Coco-Glycerides], Lipodermol®) [INCI: Octyldodecanol, Arachidyl Propionate, Tocopheryl Acetate, Retinyl Palmitate, Ethyl Linoleate, Ethyl Linolenate] or Seanamin® SU [INCI: Sorbitol, Algae Extract, Chondrus crispus (Carrageenan), Fucus Vesiculosus Extract, Algin] marketed by L. Serobiologiques/Cognis/BASF; Snow Algae Powder [INCI: Coenochloris signiensis Extract] marketed by Mibelle; Hyasol BT™ [INCI: Sodium Hyaluronate], Syn-Up™ [INCI: Benzylsulfonyl D-Seryl Homophenylalanine Amidinobenzamide Acetate] or Pentavitin® [INCI: Saccharide Isomerate] marketed by Pentapharm/DSM; Aqualance™ [INCI: Erythritol, Homarine HCl], Hydraprotectol™ [INCI: Glyceryl Polymethacrylate, Aleuritic Acid, Yeast Extract (Faex), Glycoprotein], Moist 24™ [INCI: Imperata cylindrica Root Extract], Optim Hyal™ [INCI: Hydrolyzed Yeast Extract, Cetyl Hydroxyethylcellulose, Polyglucuronic Acid], Osmocide® 4 [INCI: Glycerin, Acrylates/C10-30 Alkyl Acrylate Crosspolymer] or Revidrate™ [INCI: Ethylhexyl Palmitate, Sorbitan Oleate, Sorbitan Laureate, Myristyl Malate Phosphonic Acid] marketed by Sederma/Croda; Xpertmoist® molecular film [INCI: Glycerin, Pseudoalteromonas Ferment Extract, Xanthan Gum, Proline, Alanine, Serine, Ethylhexylglycerin, Caprylyl Glycol] or Actizyme™ GL advanced botanical ingredient [INCI: Glycerin, Mucor miehei extract, Aqua, Sodium Citrate, Potassium Sorbate, Sodium Benzoate, Phytic Acid] marketed by Lipotec/Lubrizol; and combinations thereof.

The anti-photoaging agent, and/or blue-light protector agent can be selected from the group consisting of Algaktiv Genofix™ CPD [INCI: Plankton Extract, Aqua, Lecithin] marketed by Greenaltech; Blumilight™ Biofunctional [INCI proposed: Water/Aqua (and) Butylene Glycol (and) Theobroma cacao (Cocoa) Seed Extract] marketed by Ashland; Lys′Sun™ [INCI: Hamamelis virginiana Leaf Extract, Aqua, Pentylene Glycol, Caprylyl Glycol, Xanthan Gum] marketed by BASF; Vitachelox™ [INCI: Vitis vinifera Seed Extract, Camellia sinensis Leaf Extract, Quercus robur Wood Extract] marketed by Indena; L-VCG™ [INCI: Ascorbyl Glucoside] marketed by Freshine Bio-technology; Lumicease™ blue ingredient [INCI: Glycerin, Aqua, Hydrolyzed Pea Protein, Glucose, Sodium Chloride] marketed by Lipotec/Lubrizol; Lightwaves Defense [JS+M]™ [INCI: Jasminum sambac Leaf Cell Extract] marketed by Naolys; Blue Oleoactif™ [INCI: Glycine Soja Oil, Polyglyceryl-3 Diisostearate, Oryza sativa Germ Extract, Oryza sativa Extract] marketed by Oleos-Hallstar; Majestem™ [INCI: Glycerin, Leontopodium alpinum callus Culture Extract, Xanthan Gum] or Senestem™ [INCI: Glycerin, Plantago lanceolata Leaf Extract, Xanthan Gum] marketed by Sederma; Blueshield™ [INCI: Glycerin, Capsicum Annuum Fruit Extract, Xanthan Gum] marketed by Solabia; and combinations thereof.

The DNA protecting agent, DNA repair agent, and/or stem cell protecting agent can be selected from the group consisting of; GP4G SP™ [INCI: Aqua, Glycerin, Artemia Extract], Heliostatine™ [INCI: Aqua, Glycerin, Pisum sativum Extract], Orsirtine™ [INCI: Aqua, Glycerin, Oryza sativa Extract], Chronogen™ [INCI: Aqua, Butylene Glycol, Tetrapeptide (INCI proposed)], Survixyl™ IS [INCI: Water, Butylene Glycol, Pentapeptide-31] and Chrondricare™ [INCI: Aqua, Butylene Glycol Pentapeptide-28] marketed by Vincience/ISP/Ashland; Lanacityn® [INCI: Glycerin, Aqua, Alteromonas ferment extract, Chrysanthellum indicum extract] or Melinoil™ [INCI: Isopropyl Palmitate, Lecithin, Aqua, Acetyl Hexapeptide-1] marketed by Atrium Innovations/Lucas Meyer Cosmetics; Repair Complex [INCI: Bifida Ferment Lysate] marketed by CLR; Phycojuvenine™ [INCI: Laminaria Digitata] marketed by Codif; Unirepair T-43™ [INCI: Butylene Glycol, Acetyl Tyrosine, Proline, Hydrolyzed Vegetable Protein, Adenosine Triphosphate] marketed by Induchem; Dragosine™ [INCI: Carnosine] marketed by Symrise; DN-Age [INCI: Cassia Alata Leaf Extract] marketed by Laboratories Serobiologiques/Cognis/BASF; Helioguard™ [INCI: Porphyra umbilicalis encapsulated into liposomes], PhytoCellTec™ Malus domestica [INCI: PhytoCellTec Malus domestica] or PhytoCellTec™ Argan [INCI: Argania spinosa Sprout Cell Extract, Isomalt, Lecithin, Sodium Benzoate, Aqua] marketed by Mibelle Biochemistry; Pepha-Protect™ [INCI: Water Melon Extract] marketed by Pentapharm/DSM; Celligent™ [INCI: Helianthus annuus Seed Oil, Ethyl Ferulate, Polyglyceryl-5 Trioleate, Rosmarinus officinalis Leaf Extract, Aqua, Disodium Uridine Phosphate] or Defensil™ [INCI: Octyldodecanol, Echium plantagineum Seed Oil, Cardiospermum halicacabum Extract, Helianthus annuus Seed Oil Unsaponifiables] marketed by Rahn; Venuceane™ [INCI: Thermus thermophilus Ferment, Glycerin], UV-Soft™ [INCI: Yeast Extract], Renovage™ [INCI: Caprylic/Capric Triglyceride, Teprenone], Juvinity™ [INCI: Caprylic/Capric Triglyceride, Geranylgeranyl propanol (proposed)], Phytessence™ Holyherb [INCI: Butylene Glycol, Eriodictyon californicum (Holyherb) Flower/Leaf/Stem Extract] or Resistem™ [INCI: Glycerin, Globularia cordifolia Ferment] marketed by Sederma/Croda; Infraguard™ [INCI: Caesalpinia spinosa Fruit Pod Extract, Propylene Glycol, Aqua, Helianthus annuus Sprout Extract, Sodium Benzoate, Phenoxyethanol] marketed by Mibelle; Heliomoduline™ [INCI: Low molecular weight peptides from cottonseed] or Stem-C-Guard [Hydrolyzed Pea] marketed by Silab; and combinations thereof.

The reactive carbonyl species scavenger, free radical scavengers and/or anti-glycation agent, detoxifying agent, antioxidant and/or anti-pollution agent can be selected, for example and not restricted to, from the group formed by carnosine and its derivatives; GHK™ [INCI: Tripeptide-1] and its salts and/or derivatives or Quintescine™ IS [INCI: Dipeptide-4] marketed by Vincience/ISP/Ashland; Preregen™ [INCI: Glycine soja (Soybean) Protein, Oxidoreductases], Edelweiss GC [INCI: Leontopodium alpinum Extract], Lipogard™ [INCI: Squalane, Ubiquinone], Nectapure™ [INCI: Buddleja davidii Extract, Thymus vulgaris Extract], Alpaflor Nectapure™ [INCI: Buddleja davidii Extract, Thymus vulgaris Extract, Glycerin, Water] or Dismutin-BT™ [INCI: Highly purified SOD from a natural yeast strain of Saccharomyces cerevisiae] marketed by Pentapharm/DSM; Preventhelia® [INCI: Diaminopropionoyl Tripeptide-33], Aldenine® [INCI: Hydrolyzed Wheat Protein, Hydrolyzed Soy Protein, Tripeptide 1], Lipochroman™ [INCI: Dimethylmethoxy Chromanol], Thermostressine® [INCI: Acetyl Tetrapeptide-22] Pollushield™ functional ingredient [INCI: Diisopropyl Adipate, Lecithin, Acrylic Acid/Acrylamidomethyl Propane Sulfonic Acid Copolymer, Dimethylmethoxy Chromanol, Xanthan Gum] or Bodyfensine® [INCI: Acetyl Dipeptide-3 Aminohexanoate] marketed by Lipotec/Lubrizol; Sunactyl™ [INCI: Mannitol, Pisum sativum Extract, Histidine HCl, Arginine, Cyclodextrin, Dextrin, Yeast Extract, Acetyl Tyrosine, Pyridoxine HCl, Khaya Senegalensis Bark Extract, Nicotinamide, Adenine Dinucleotide, Disodium Succinate, Aspartic Acid], Imidinyl™ [INCI: Tamarindus Indica Seed Polysaccharide], Phystrogene™ [INCI: Butylene Glycol, Malva sylvestris (Mallow) Extract, Xanthan Gum] or Purisoft™ [INCI: Moringa Pterogysperma Seed Extract] marketed by Laboratoires Sérobiologiques/Cognis/BASF; AquaCacteen™ [INCI: Glycerin, Opuntia Ficus Indica Stem Extract, Phenoxyethanol, Aqua], Trimoist™ (KMF) [INCI: Sodium Stearoyl Lactylate, Cetyl alcohol, Olus Vegetable oil, Tocopheryl acetate, Glycerin, Glycine soja sterol, Sodium lactate, Sodium carboxymethyl beta-glucan, Carnosine, Lactic Acid], MelanoBronze™ [INCI: Vitex agnus castus Extract (Monk's pepper berries extract (phyto-endorphins)), Acetyl Tyrosine], CM-Glucan™ [INCI: Sodium Carboxymethyl Beta-glucan, Phenoxyethanol, SunActin™ [INCI: Helianthus annuus (Sunflower) Sprout Extract, Tocopherols, Glycerin, Lecithin, Phenoxyethanol, Aqua], GSP-T™ skin [INCI: Glycerin, Alcohol, Aqua, PEG-40 Hydrogenated Castor Oil, Vitis vinifera (Grape) Seed Extract] or Detoxophane™ [INCI: Lepidium sativum Sprout Extract, Lecithin, Phenoxyethanol, Glycerin, Water] marketed by Mibelle Biochemistry; Bacocalmine™ [INCI: PEG-8, Bacopa Monnieri Extract, Water (Aqua), Hydroxyethylcellulose], Kombuchka™ [INCI: Saccharomyces/Xylinum Black Tea Ferment, Glycerin, Hydroxyethyl cellulose], Citystem™ [INCI: Glycerin, Marrubium vulgare Extract] or Prodizia™ [INCI: Albizia julibrissin Extract, Glycerin] marketed by Sederma/Croda; Extramel™ C [INCI: Hydroxypropyltrimonium Maltodextrin Crosspolymer, Cucumis melo (Melon) Fruit Extract] marketed by Seppic; Defensine™ [INCI: Triticum vulgare Germ Extract], Apolluskin® [INCI: Taraxacum officinale (Dandelion) Extract], Detoxyl® [INCI: Water, Butylene Glycol, Butyrospermum parkii (Shea Butter) Seedcake Extract] or Antiglyskin™ [INCI: Aqua, Helianthus annuus Seed Extract] marketed by Silab; and combinations thereof.

The compositions of the invention may also include other active ingredients such as vitamins, minerals, proteins, peptides, fatty acids, antioxidants, anti-inflammatory agents, darkening agents and/or mixtures thereof. Particularly, the cosmetic composition may include other active ingredients for promoting hair growth, and/or preventing hair loss. Suitable, non-limiting examples, in the context of this invention are Growth Oleoactif® [INCI: Helianthus annuus (Sunflower) Seed Oil (and) Polyglyceryl-3 Diisostearate (and) Carthamus tinctorius (Safflower) Flower Extract (and) Hibiscus sabdariffa Flower Extract] marketed by Hallstar. Widelash™ [INCI: Glycerin, Water (Aqua), Panthenol, Biotinoyl Tripeptide-1] marketed by Sederma; SymPeptide® Xlash [INCI: Glycerin (and) Aqua (and) Myristoyl Pentapeptide-17], SymLash® 1631 [INCI: Pentylene Glycol (and) Isochrysis galbana Extract] marketed by Symrise; Anargy™ [INCI: Water (and) Butylene Glycol (and) Oligopeptide-2 (and) Nicotiana benthamiana Hexapeptide-40 sh-Polypeptide-9 (and) Nicotiana benthamiana Hexapeptide-40 sh-Polypeptide-86] marketed by Lipotrue; Capixyl™ [INCI: Butylene Glycol (and) Aqua (and) Dextran (and) Acetyl Tetrapeptide-3 (and) Trifolium pratense (Clover) Flower Extract] marketed by Lucas Meyer; Nano Lashes™ [INCI: Aqua, Simmondsia chinensis Seed Extract, Polysorbate 20, Hydroxypropyl guar, Sodium Benzoate, Potassium Sorbate] marketed by Nanovetores; Specped SC-MH16™ [INCI: Aqua, Glycerin, Myristoyl Hexapeptide-16, Caprylyl Glycol, Ethylhexylglycerin], Myristoyl Pentapeptide-17 [INCI: Myristoyl Pentapeptide-17], SpecPed® BT1 [INCI: Biotinoyl Tripeptide-1], SpecPed® LashLD [INCI: Biotinoyl Tripeptide-1, Glycerin, Water, Caprylyl Glycol & Ethylhexylglycerin, Panthenol, Trehalose], SpecPed® MP17P [INCI: Myristoyl Pentapeptide-17] marketed by Spec-chem; Procapil™ [INCI: Butylene Glycol (and) Aqua (and) PPG-26-Buteth-26 (and) PEG-40 Hydrogenated Castor Oil (and) Apigenin (and) Oleanolic Acid (and) Biotinoyl Tripeptide-1] marketed by Sederma; AnaGain™ [INCI: Pisum sativum (Pea) Sprout Extract (and) Isomalt (and) Aqua], PhytoCellTec™ Malus domestica Hair [INCI: Malus domestica Fruit Cell Culture Extract (and) Xanthan Gum (and) Glycerin (and) Lecithin (and) Phenoxyethanol (and) Aqua/Water], Santenergy™ [INCI: Bioflavonoids (and) Pentylene Glycol (and) Alcohol (and) Aqua], RootBioTec™ HW [INCI: Ocimum basilicum Hairy Root Culture Extract (and) Alcohol (and) Aqua/Water], RootBioTec™ HO [INCI: Ocimum basilicum Hairy Root Culture Extract (and) Helianthus annuus (Sunflower) Seed Oil (and) Cocos nucifera (Coconut) Oil] marketed by Mibelle; CAPILIA LONGA PPF [INCI: Curcuma longa (Turmeric) callus Culture Conditioned Media (and) Water] marketed by Vytrus Biotech; Cressatine® [INCI: Glycerin (and) Aqua (and) Nasturtium officinale Extract (and) Tropaeolum majus Extract] marketed by Solabia; Alotide™ [INCI: Copper Ascorbyl Phosphate Succinyl Tripeptide-34] marketed by Peptron; BURGEON-UP™ [INCI: Aqua (and) Alcohol (and) Nasturtium officinale Leaf/Stem Extract] marketed by Ichimaru Pharcos; Capalgin® [INCI: Chondrus crispus Extract] marketed by Exysmol; Dermosaccharides® GY [INCI: Aqua (and) Glycerin (and) Glycogen (and) Phenoxyethanol (and) Methylparaben], Trichogen™ VEG LS 9922 [INCI: Water (and) Panax ginseng Root Extract (and) Arginine (and) Acetyl Tyrosine (and) Arctium majus Root Extract (and) Hydrolyzed Soy Protein (and) Polyquaternium-11 (and) PEG-12 Dimethicone (and) Calcium Pantothenate (and) Zinc Gluconate (and) Niacinamide (and) Ornithine HCl (and) Citrulline (and) Glucosamine HCl (and) Biotin] marketed by BASF; Follicusan™ DP [INCI: Aqua (and) Alcohol Denat. (and) Panthenyl Ethyl Ether (and) Inositol (and) Milk Protein (and) Lactose (and) Acetyl Cysteine (and) Acetyl Methionine (and) Sodium Citrate (and) Citric Acid] marketed by CLR; Hairline® [INCI: Propanediol (and) Water (aqua) (and) Lindera strychnifolia Root Extract] marketed by Greentech; Kerascalp™ [INCI: Propanediol (and) Glycerin (and) Phyllanthus Emblica Fruit Extract], Baicapil™ [INCI: Propanediol (and) Water (and) Arginine (and) Lactic Acid (and) Glycine soja (Soybean) Germ Extract (and) Triticum vulgare (Wheat) Germ Extract (and) Scutellaria baicalensis Root Extract (and) Sodium Benzoate (and) Gluconolactone (and) Calcium Gluconate] marketed by Provital; BIOENERGIZER™ P BG PF [INCI: Aqua/Water, Butylene Glycol, Panthenol, Propylene Glycol, Pelvetia Canaliculata Extract, Laminaria digitata Extract] marketed by Seppic; Protectagen™ [INCI: Aqua (and) Glycerin (and) Hydrolyzed Rice Protein] marketed by Ashland; Redensyl® [INCI: Glycerin (and) Aqua (and) Sodium Metabisulfite (and) Larix europaea Wood Extract (and) Glycine (and) Zinc Chloride (and) Camellia sinensis Leaf Extract] marketed by Givaudan; GANOTHER® [INCI: Glycerin (and) Water (and) Ganoderma lucidum (Mushroom) Mycelium Ferment Filtrate] marketed by B&G; Hairgenyl® [INCI: Saccharomyces cerevisiae Extract], Anageline® [INCI: Hydrolyzed Lupine Protein] marketed by Silab; Hairdian™ AP [INCI: Propanediol (and) Thuja orientalis Extract (and) Zingiber officinale (Ginger) Root Extract (and) Trifolium pratense (Clover) Leaf Extract (and) Artemisia argyi Leaf Extract] marketed by Shanghai GREAF Biotech, Naturein™ Wheat Peptides (INCI: Water (aqua) (and) Hydrolyzed Wheat Protein) or from Givaudan, KeraMatch™ V (INCI: Aqua (and) Hydrolyzed Pea Protein (and) Hydrolyzed Vegetable Protein) from Croda Personal care.

### Applications

The present invention is based on the finding that the protein hydrolysate of the invention is effective in alleviating and/or preventing adverse effects of the exposome on skin and/or hair. Adverse effects of the exposome include adverse effects due to air pollution, sunlight and/or greenhouse gases. It has also been found that the protein hydrolysate is effective in alleviating adverse effects of chemical treatment or heat stress on hair. In addition, it has been found that the protein hydrolysate is effective in maintaining and/or improving the physical barrier function of the skin. The physical barrier function of the skin is also referred to herein as the physical barrier of the skin, the skin's permeability barrier or simply the skin barrier. This barrier is provided by the stratum corneum and the tight junctions in the epidermis.

In one aspect, the invention provides for the use of the protein hydrolysate described herein, or a compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg,

and
stereoisomers, and cosmetically acceptable salts thereof, or a combination of said protein hydrolysate or said compound and a filtrate obtained by the fermentation of Raphanus sativus root by the microorganism Leuconostoc, for the treatment and/or care of the skin and/or hair. In particular, the treatment and/or care of the skin and/or hair is cosmetic and non-therapeutic.

The use of the protein hydrolysate, compounds or combinations described herein can be for the alleviation and/or prevention of adverse effects of the exposome on hair; the alleviation and/or prevention of adverse effects of air pollution on hair; the alleviation and/or prevention of adverse effects of sunlight on hair; the alleviation and/or prevention of the adverse effects of chemical stress on hair; the alleviation and/or prevention of adverse effects of heat stress on hair; and/or the alleviation or prevention of the adverse effects to the hair caused by peroxidation of lipids in hair and/or carbonylation of proteins in hair. The use of the protein hydrolysate, compounds or combinations described herein can be for the alleviation and/or prevention of adverse effects of air pollution and sunlight on hair. Adverse effects in hair can manifest themselves in the form of a damaged hair. Thus, the protein hydrolysate can be used for reducing hair damage. Adverse effects in hair can manifest themselves in the form of a hair with more split ends; reduced fiber alignment and/or increase in frizz. Therefore, the hair is less manageable. Thus, the protein hydrolysate can be used for increasing hair manageability, particularly by a reduction of split ends and/or an increase in fiber alignment and/or reduction of frizz and/or a reduction of volume.

Thus, there is provided the use of the protein hydrolysate, compounds or combinations described herein for the cosmetic, non-therapeutic treatment and/or care of the hair, wherein the treatment and/or care of the hair is the alleviation or prevention of the adverse effects of the exposome on the hair.

There is provided the use of the protein hydrolysate, compounds or combinations described herein for the cosmetic, non-therapeutic treatment and/or care of the hair, wherein the treatment and/or care of the hair is the alleviation or prevention of the adverse effects of the air pollution on the hair, in particular, where the air pollution contains particulate matter.

Air pollution includes particulate matter, for example. Particulate matter is an airborne pollutant that includes solid particles. Particulate matter may include dust, dirt, soot or smoke. Particulate matter can come from both anthropogenic and naturally occurring sources, including automobiles (e.g., exhaust emissions from combustion engines of automobiles), power plants, factories, fires, wood-burning stoves, dust from open land or construction, dust from roads (e.g., brake-, tire- and road-wear particles), pollen (e.g., cedar pollen) and smoke from tobacco (e.g., cigarette smoke). The particulate matter can have a size of 10 microns or less (also referred to as PM₁₀ and “dust”). The particulate matter can arise from automobile combustion engines (e.g., diesel particulate such as those encountered in Diesel particulate matters NIST1650B. The particulate matter can comprise pollen (e.g., cedar pollen).

There is provided the use of the protein hydrolysate, compounds or combinations described herein for the cosmetic, non-therapeutic treatment and/or care of the hair, wherein the treatment and/or care of the hair is the alleviation or prevention of the adverse effects of the air pollution containing particulate matter on the hair, where the particulate matter is derived from road dust, derived from diesel, or is pollen.

There is provided the use of the protein hydrolysate, compounds or combinations described herein for the cosmetic, non-therapeutic treatment and/or care of the hair, wherein the treatment and/or care of the hair is the alleviation or prevention of the adverse effects of sunlight on the hair. The sunlight can comprise UV-A radiation.

There is provided the use of the protein hydrolysate, compounds or combinations described herein for the cosmetic, non-therapeutic treatment and/or care of the hair, wherein the treatment and/or care of the hair is the alleviation or prevention of the adverse effects of bleaching or dyeing on hair.

There is provided the use of the protein hydrolysate, compounds or combinations described herein for the cosmetic, non-therapeutic treatment and/or care of the hair, wherein the treatment and/or care of the hair is the alleviation or prevention of the adverse effects of heat stress on hair.

There is provided the use of the protein hydrolysate, compounds or combinations described herein for the cosmetic, non-therapeutic treatment and/or care of the hair, wherein the treatment and/or care of the hair is the alleviation or prevention of the adverse effects to the hair caused by peroxidation of lipids in hair and/or carbonylation of proteins in hair.

There is provided the use of the protein hydrolysate, compounds or combinations described herein for the cosmetic, non-therapeutic treatment and/or care of the hair, wherein the treatment and/or care of the hair is the increase of hair manageability; and/or the increase in fiber alignment and/or reduction of frizz and/or the reduction of volume.

The use of the protein hydrolysate, compounds or combinations described herein can be for the alleviation and/or prevention of adverse effects of the exposome on skin; the alleviation and/or prevention of adverse effects of air pollution on skin; the alleviation and/or prevention of adverse effects of sunlight on skin; the alleviation and/or prevention of adverse effects caused by greenhouse gases; and/or the maintenance and/or improvement of the physical barrier function of the skin. The use of the protein hydrolysate, compounds or combinations described herein can be for the alleviation and/or prevention of adverse effects of air pollution and sunlight on skin. Adverse effects to skin can manifest themselves in the form of a reduction in barrier function; a dull appearance of the skin; a loss of brightness, luminosity and/or glossiness of the skin; a loss in homogeneity in the texture of the skin; an increase in roughness of the skin; the appearance of dark spots; and/or erythema (skin redness).

Thus, there is provided the use of the protein hydrolysate, compounds or combinations described herein for the cosmetic, non-therapeutic treatment and/or care of the skin, wherein the treatment and/or care of the skin is the alleviation or prevention of the adverse effects of the exposome on the skin.

There is provided the use of the protein hydrolysate, compounds or combinations described herein for the cosmetic, non-therapeutic treatment and/or care of the skin, wherein the treatment and/or care of the skin is the alleviation or prevention of the adverse effects of the air pollution containing particulate matter on the skin, where the particulate matter is derived from road dust, derived from diesel, or is pollen.

There is provided the use of the protein hydrolysate, compounds or combinations described herein for the cosmetic, non-therapeutic treatment and/or care of the skin, wherein the treatment and/or care of the skin is the alleviation or prevention of the adverse effects of sunlight on the skin. The sunlight can comprise UV-A radiation.

Adverse effects can manifest themselves in the form of a loss in barrier function in the skin. Thus, there is provided the use of the protein hydrolysate, compounds or combinations described herein for the cosmetic, non-therapeutic treatment and/or care of the skin, wherein the treatment and/or care of the skin is the maintenance and/or improvement of the physical barrier function of the skin.

Adverse effects can manifest themselves in the form of skin having a dull appearance; and/or having a less bright appearance; and/or having a less luminous appearance and/or a less glossy appearance. Thus, there is provided the use of the protein hydrolysate, compounds or combinations described herein for the cosmetic, non-therapeutic treatment and/or care of the skin, wherein the treatment and/or care of the skin is increasing skin brightness, and/or increasing the gloss of the skin and/or increasing skin luminosity.

The adverse effects may also lead to the appearance of dark spots. Thus, there is provided the use of the protein hydrolysate, compounds or combinations described herein for the cosmetic, non-therapeutic treatment and/or care of the skin, wherein the treatment and/or care of the skin is reducing dark spots. In particular, the treatment and/or care can involve reducing melanin in the skin.

The adverse effects can manifest themselves in the form of a reduction of skin tone and/or skin texture homogeneity and/or an increase of skin roughness. Thus, there is provided the use of the protein hydrolysate, compounds or combinations described herein for the cosmetic, non-therapeutic treatment and/or care of the skin, wherein the treatment and/or care of the skin is increasing skin tone and/or increasing the homogeneity of skin texture and/or reducing skin roughness.

The adverse effects can manifest themselves in the form of erythema or skin redness. Thus, there is provided the use of the protein hydrolysate, compounds or combinations described herein for the cosmetic, non-therapeutic treatment and/or care of the skin, wherein the treatment and/or care of the skin is reducing skin redness.

In one aspect, the invention relates to a method of treatment and/or care of the skin and/or hair comprising administering a protein hydrolysate of azuki beans according to the first aspect of the invention, or a compound chosen from:

(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg,

and
stereoisomers, and cosmetically acceptable salts thereof, or a combination of said compound and a filtrate obtained by the fermentation of Raphanus sativus root by the microorganism Leuconostoc to a subject. The treatment and/or care may be a cosmetic, non-therapeutic treatment and/or care. The protein hydrolysate, compound or combination described above may be present in a cosmetic composition, for example a cosmetic composition as described herein. The method can be for the treatment and/or care of the skin and/or hair as described above in relation to applications (uses) of the protein hydrolysate, compounds and/or combinations of the invention.

The treatment and/or care is the alleviation or prevention of the adverse effects of the exposome on the skin and/or hair; the alleviation or prevention of the adverse effects of air pollution on the skin and/or hair; the alleviation or prevention of the adverse effects of sunlight on the skin and/or hair; the alleviation or prevention of the adverse effects of chemical treatment of the hair; the alleviation or prevention of the adverse effects of heat stress on the hair; and/or the maintenance and/or improvement of the physical barrier function of the skin.

In the applications of the protein hydrolysate, compounds, and combinations of the invention, the treatment and/or care can be preventative in that it serves to protect the skin and/or hair from adverse effects. In this case, the protein hydrolysate, compounds, and combinations of the invention are applied to the skin and/or hair prior to exposure of the stressor. The treatment and/or care can be reparative or restorative, i.e., where alleviation has a reparative or restorative function, (e.g., with respect to aesthetic properties). In this case, the protein hydrolysate, compounds, and combinations of the invention can be applied to the skin and/or hair during or after exposure of the stressor.

For the above-described methods of the invention, topical or transdermal application can be carried out by iontophoresis, sonophoresis, electroporation, mechanical pressure, osmotic pressure gradient, occlusive cure, microinjections, by needle-free injections by means of pressure, by microelectric patches, face masks or any combination thereof.

For the above described methods of the invention, the frequency of application or administration can vary greatly, depending on the needs of each subject, with a recommendation of an application from once a month to ten times a day, preferably from once a week to four times a day, more preferably from three times a week to twice a day, even more preferably once a day.

The invention also provides for the use of the protein hydrolysate of the invention in a therapeutic treatment of the skin. Thus, the invention provides a protein hydrolysate as disclosed herein for use as a medicament. In particular, the invention provides a protein hydrolysate as disclosed herein for use in the prevention or treatment of a disease or disorder associated with the exposome, exposure of the skin to air pollution, sunlight and/or greenhouse gases, and/or associated with an impaired physical barrier function of the skin. Further, the invention provides for the use of a compound of formula (I), its stereoisomers and/or its pharmaceutically acceptable salts for the manufacture of a medicament for the treatment or prevention of a disease or disorder. The invention is further defined by the following non-limiting examples.

### Example 1

### Preparation of Ground Azuki Beans

The first step was the processing of the raw beans into a fine powder of brown granulated appearance. The aim of this stage is to reduce particle size as much as possible in order to facilitate protein solubilization. The azuki beans are ground into a flour and larger fragments are sifted out.

### Example 2

### Enzymatic Hydrolysis

### 2.1 Pre-Treatment of the Flour

Preliminary treatment of the ground azuki beans (also referred to herein as “flour”) increases the content of water-soluble protein in the hydrolysis media, prior to enzyme addition. The flour is suspended in deionized water at a concentration of 21.8 g of flour/Kg of water (w/w) and transferred into a suitable reactor vessel equipped with pH and temperature probes providing real-time monitoring. Initial pH is raised from 6.5-6.8 to 7.0 by addition of a basic solution (i.e., NaOH 1N) and, when needed, more solvent is added to reach a desired concentration. The conditions are maintained for 1 hour at room temperature (25±2° C.). The resulting suspension is referred to as the “protein suspension”—it is a suspension of ground azuki beans in water containing proteins from the ground azuki beans that have been solubilized.

### 2.2. Enzymatic Hydrolysis

Enzyme is added to the protein suspension and the mixture stirred for 1 hour at room temperature (25±2° C.). Agitation conditions are adjusted to ensure homogenous and vigorous stirring, while keeping foam formation at minimum levels. Then, Alcalase™ is added to the protein suspension. Alcalase™ is a subtilisin type enzyme. Preferred hydrolysis conditions are defined by the use of subtilisin-type Alcalase™ AF 2.4 L (CAS no. 9014-01-1, EC 3.4.21.62, Novozymes®) enzymes.

Appropriate progress of hydrolysis is monitored by changes in pH. A drop in pH is associated with the release of peptide fragments and subsequent buildup of acidic carboxylic acids. Typically, crude hydrolysate suffers slight acidification during the process (0.2-0.3 drop in pH units).

### 2.3 Enzyme Inactivation

After enzymatic hydrolysis, the enzyme is inactivated by increasing temperature to at least 70° C. and maintaining the temperature for at least 20 min. The hydrolysate obtained is hereinafter referred to as “crude hydrolysate”.

### Example 3

### Purification

With the aim of enriching the mixture in the desired active components (i.e., peptides) further clarification and dialysis steps can be performed as described below in more detail.

### 3.1. Centrifugation.

Hydrolyzed Azuki suspension (“crude hydrolysate”) is optimally centrifugated in continuous flow (i.e., using Westfalia CSA-1 centrifuge or similar equipment) at 10.000×G, with an optimal flow of 50 l/h. Supernatant is collected, whereas pellet was discarded as waste material. Supernatant turbidity is controlled throughout the operation taking a representative sample, and maintained below 0.2 AU/g of protein at 600 nm (relative to an appropriate blank in these conditions).

### 3.2. Frontal Filtration.

The supernatant obtained from step 3.1. is passed through filters of varying size under pump-generated pressure. First, a 1.2 μm pore filter is used to discard the bulk of large particles that were not eliminated during centrifugation. This first filtration step is optional, and the purpose is to reduce the number of small pore size filters needed. Then, the obtained filtrate is subsequently filtered through 0.45 and 0.22 μm pore-sized filters.

### 3.3. Ultrafiltration.

The filtrate obtained in Example 3.2. is further ultrafiltrated through an ultrafiltration membrane (Millipore ref. P2PLBCC05, 0.5 m²/cassette) to remove fragments with molecular weight higher than 3 KDa. The resulting filtrate obtained is also referred to as “purified hydrolysate” or “permeate”.

### Example 4

### Purified Hydrolysate Characterization

Twelve different batches of purified hydrolysate obtained as described from Examples 1 to 3 are analyzed to determine the dry weight, protein content and antioxidant activity.
Dry weight is determined by weight difference after drying at 110° C.
Protein content is determined by using a BCA Assay kit.
Antioxidant activity is determined by DPPH (2,2-Diphenyl-1-Picrylhydrazyl) assay.
Briefly, 50 μL of 100 UM DPPH in EtOH is mixed with 50 μL of purified hydrolysate and incubated in the dark at 37° C. for 20 min and absorbance measured at 517 nm.

The inhibition percentage is calculated from the following formula:

$$Inhibition ( \% ) = \frac{( Blank Absorbance - Sample Absorbance )}{Blank absorbance} \times 100$$

Polyphenol content as determined by Folin-Ciocalteu method.

The dry weight of the different batches was on average from 2 to 5 mg of dry weight/purified hydrolysate. The protein content ranged from 19 to 35% (w/w). DPPH activity ranged from 74 to 80%. Polyphenol content was from 0.15 to 0.30 mg/mL.

### Example 5

In Vitro Repair Effect on Follicle Dermal Papilla Cells from Scalp

Human Follicle Dermal Papilla Cells (HFDPC) derived from scalp are cultivated with Follicle Dermal Papilla Cell Growth Medium at 37° C. and 5% CO₂. These cells are stressed with Particle Matters (PM) at 0.1 μg/cm² (from European Reference Material; Ref. CZ100) followed by UV-A irradiation (LED source, emission peak at 365 nm, 3 J/cm²) in 1×PBS buffer (Phosphate Buffered Saline, pH 7.4). The cells are treated with the stressors for 13 min. Just after stress exposure, cells are treated with purified hydrolysate of Example 3 at 1% (v/v) in culture medium. The antioxidant N-acetyl-cysteine (NAC) is used as a positive control. Two (2) hours after the intracellular levels of protein carbonylation are measured.

### Analysis of Protein Carbonylation:

Carbonylated proteins in hair shafts or HFDPC are labeled in situ using a fluorescent probe functionalized to specifically bind carbonyl groups in proteins [Baraibar, M A, et al., “Proteomic quantification and identification of carbonylated proteins upon oxidative stress and during cellular aging” J. Proteomics (2013) 30; 92:63-70]. Specific fluorescent signals (relative fluorescent units) per experimental group (n=6) are collected and analyzed with ImageJ™ software. A relative value (%) of Carbonylation Level per experimental group is obtained with respect to basal control.

Repair value (%) is obtained for the experimental groups accordingly to the following formula:

$$Repair value = [ ( Carbonylation Stress - Carbonylation group X ) / ( Carbonylation Stress - Carbonylation Control ) ] \times 100$$

The control group is considered at maximum efficiency (100%) and the stress group at minimum efficiency (at 0%):

TABLE 1

| Exp. Group | Mean ± SEM | Repairing efficacy (%) |
|---|---|---|
| Control | 100 ± 3.3 | 100% |
| Stress (PM + UV-A) | 126.9 ± 3.0 | 0% |
| Azuki 1% (v/v) + Stress | 119.4 ± 3.0 | 28% |

The purified hydrolysate significantly repaired and protected from the deleterious effect of particle matters and UV-A radiation.

### Example 6

In Vitro Protection Evaluation on Follicle Dermal Papilla Cells from Scalp

Human Follicle Dermal Papilla Cells (HFDPC) derived from scalp are cultivated with HFDPC Growth Medium at 37° C. and 5% CO₂. These cells are treated with purified hydrolysate according to Example 3 at 1% (v/v) in culture medium for 24 hours at 37° C. and 5% CO₂ (prior to stress). The next day, cells are stressed with Particle Matters at 0.1 μg/cm² (from European Reference Material; Ref.CZ100) and Cedar pollen (Ref. Cedar Pollen-Cj*; LG-5280 Euromedex™; 5 ng/cm²) followed by UV-A irradiation (LED source, emission peak at 365 nm, 3 J/cm²) in 1×PBS buffer (Phosphate Buffered Saline, pH 7.4). Stress treatment is conducted for 13 minutes. After treatments, cells are fixed and carbonyl protein labeled in situ. The detection of protein oxidation (carbonylation) is performed by epifluorescence microscopy on fixed cells.

Just after treatments and stress exposure, cells (HFDPC) are fixed on the plate with an Ethanol/Acetic Acid solution. Carbonylated proteins are labeled in situ using a fluorescent probe (Ex=647 nm/Em=650 nm) functionalized to specifically bind carbonyl groups. Fluorescent images are collected with an epifluorescence microscope (ThermoFisher, Evos M5000) and analyzed with ImageJ™ software (Schneider, 2012). Image collection for the different conditions is achieved using identical conditions of acquisition (40× objective).

The raw images are collected in a range of intensity of specific signal from 0 to 65535 and analyzed with ImageJ™ software. The intensity of carbonylation is determined by integration of the specific fluorescence signal normalized by the evaluated area.

An efficacy value (%) is obtained for the experimental groups accordingly to the following formula:

$$Efficacy \% of sample = [ ( Oxidation level Stress - oxidation level in sample tested ) / ( oxidation level Stress - oxidation level Control ) ] \times 100$$

As reference, the control group is considered the maximum efficiency (100%) and the stress group the minimum efficiency (at 0%):

TABLE 2

| | | Protection |
|---|---|---|
| Exp. Group | Mean ± SEM | efficacy (%) |
| Control | 100 ± 0.4 **** | 100% |
| Stress (PM + UV-A + Cedar pollen) | 130 ± 2.9 | 0% |
| Azuki 1% (V/V) + Stress | 117 ± 1.0** | 43% |

p-Values are obtained by ANOVA variance analysis followed by Dunnett's post-hoc test for multi-comparisons in comparison with stress.
**** p-Value < 0.0001,
*** p-Value < 0.001,
**p-Value < 0.01,
* p-Value < 0.05

The purified hydrolysate protected from the adverse effects (carbonylation) caused by stressors.

### Example 7

### Ex Vivo Protection Evaluation in Caucasian Hair Shafts (Ex Vivo)

Caucasian hair shafts (female donor; light brown) are treated with hydrolysate of Example 3 at 1% (v/v) (active ingredient solubilized in ultra-pure water, leave on (no washing steps)) for 30 minutes with gently movement at room temperature. After this time, hair shafts are dried at room temperature and are stressed with Particle Matters at 150 μg/cm² (from European Reference Material; Ref. CZ100) followed by UV-A irradiation (LED source, emission peak at 365 nm, 84 J/cm²) for 6 h.

After stress exposure Proteins Carbonylation is determined as described in Example 5.

TABLE 3

| Carbonylation level % | MEAN ± SEM | Protection efficacy |
|---|---|---|
| BASAL CONTROL | 100 ± 8.06 **** | 100% |
| STRESS NON-TREATED | 214 ± 9.01 | 0% |
| AZUKI 1% (v/v) (STRESS) | 149.8 ± 5.71 *** | 56.34% |
| Positive control NAC (stress) | 166.8 ± 5.41** | 41.39% |

p-Values were obtained by ANOVA variance analysis followed by Dunnett's post-hoc test for multi-comparisons in comparison with stress.
**** p-Value < 0.0001;
*** p-Value < 0.001;
**p-Value < 0.01

Results confirm the protective effect of purified hydrolysates.

### Example 8

### Ex Vivo Protection Evaluation in Asian Hair Shafts

Asian hair shafts, are treated with purified hydrolysate according to Example 3 at 1% (v/v) (active ingredient solubilized in ultra-pure water, leave on (no washing steps)) for 30 minutes with a gently movement at room temperature. After this time, hair shafts are dried at natural evaporation at room temperature and are stressed with Cedar pollen (Ref. Cedar Pollen-Cj*; LG-5280 Euromedex™; 500 ng/cm²), followed by Particulate Matter application (150 μg/cm², applied as powder; from European Reference Material; Ref. ERM-CZ100) and UV-A irradiation (LED source, emission peak at 365 nm, 84 J/cm²; 6 hours of irradiation).

After stress exposure protein carbonylation is measured. Hair shafts from each experimental condition are sampled, cryo-preserved in Cryomatrix™ (OCT), snap-frozen into liquid nitrogen and kept at −80° C. until OxiProteome™ View analysis. Hair shaft cross-sections (perpendicular to the hair axis, sagittal view) of 3 μm of thickness are obtained using a cryostat (Leica) for the analysis. For lateral (cuticle) view, carbonylated proteins are labeled on entire hair shafts. Carbonylated proteins are labeled in situ using a fluorescent probe (Ex=647 nm/Em=650 nm) functionalized to specifically bind carbonyl groups. Fluorescent images are collected with an epifluorescence microscope (ThermoFisher, Evos M5000) and analyzed with ImageJ™ software (Schneider, 2012). Image collection for the different conditions is achieved using identical conditions of acquisition (40× objective). The raw images were collected in a range of intensity of specific signal from 0 to 65535 and analyzed with ImageJ™ software. The intensity of carbonylation is obtained by the integration of the specific fluorescence signal normalized by the evaluated area. For hair shaft cross-sections, in each image, the quantification of carbonylation level is independently obtained for the different compartments (cuticle or cortex) and for the whole hair. Three (3) images per condition were used to quantify the carbonylation levels; the mean value and standard deviation are obtained. Data treatment and statistical analysis are achieved by using graphPad Prism™ (La Jolla, California, USA).

An efficacy value (%) is obtained for the experimental groups according to the following formula.

Efficacy % (test group x)=[Oxidation level (Stress)−Oxidation level (group X)]/[Oxidation level (Stress)−Oxidation level (Control)*100

As reference, the control group is considered the maximum efficiency (100%) and the stress group the minimum efficiency (at 0%):

TABLE 4
WHOLE HAIR

| Exp. Group | Mean ± SEM | Protection efficacy (%) |
|---|---|---|
| Control | 100 ± 10.2 **** | 100% |
| Stress (PM + UV-A) | 215 ± 4.1 | 0% |
| Azuki 1% + Stress | 114 ± 2.9 **** | 88% |

CUTICLE

| Exp. Group | Mean ± SEM | Protection efficacy (%) |
|---|---|---|
| Control | 100 ± 15.5 *** | 100% |
| Stress (PM + UV-A) | 250 ± 3.2 | 0% |
| Azuki 1% + Stress | 164 ± 13.1 ** | 57% |

CORTEX

| Exp. Group | Mean ± SEM | Protection efficacy (%) |
|---|---|---|
| Control | 100 ± 5.7 *** | 100% |
| Stress (PM + UV-A) | 190 ± 10.1 | 0% |
| Azuki 1% + Stress | 83 ± 3.8 **** | 100% |

p-Values were obtained by ANOVA variance analysis followed by Dunnett's post-hoc test for multi-comparisons in comparison with stress.
**** p-Value <0.0001
*** p-Value < 0.001
** p-Value < 0.01
*p-Value < 0.05

### Example 9

### Ex Vivo Protection Evaluation in African Hair Shafts

African hair shafts are treated with Azuki hydrolysate of Example 3 at 1% (v/v) (active ingredient solubilized in ultra-pure water, leave on (no washing steps)) during 30 minutes in a gently movement at room temperature. After this time, hair shafts are dried at room temperature and are stressed with Particulate Matter application (150 μg/cm², applied as powder; from European Reference Material; Ref. ERM-CZ100) and UV-A irradiation (LED source, emission peak at 365 nm, 84 J/cm²). The stress treatment is conducted for 6 h.

After stress exposure Proteins Carbonylation is measured as determined in Example 8.

TABLE 5
WHOLE HAIR

| Exp. Group | Mean ± SEM | Protection efficacy (%) |
|---|---|---|
| Control | 100 ± 2.0**** | 100% |
| Stress (PM + UV-A) | 159 ± 5.7 | 0% |
| Azuki 1% + Stress | 131 ± 2.9** | 46% |

CUTICLE

| Exp. Group | Mean SEM | Protection efficacy (%) |
|---|---|---|
| Control | 100 ± 3.2**** | 100% |
| Stress (PM + UV-A) | 177 ± 3.5 | 0% |
| Azuki 1% + Stress | 148 ± 4.6** | 38% |

CORTEX

| Exp. Group | Mean ± SEM | Protection efficacy (%) |
|---|---|---|
| Control | 100 ± 2.2** | 100% |
| Stress (PM + UV-A) | 138 ± 0.9 | 0% |
| Azuki 1% + Stress | 121± 8.1^{IS} | 44% |

p-Values are obtained by ANOVA variance analysis followed by Dunnett's post-hoc test for multi-comparisons in comparison with stress.
****p-Value < 0.0001;
*** p-Value < 0.001;
****p-Value <0.0001;
**p-Value < 0.01;
* p-Value < 0.05;
^{IS}p-Value < 0.10

### Example 10

### Ex Vivo Defense Evaluation in Latin American Hair Shafts

Latin American curly hair shafts are treated with a commercial dye product (chocolate color) with Azuki purified hydrolysate of Example 3 at 1% (v/v) (active ingredient solubilized in the dying product) for 30 minutes of contact.

In situ detection of fluorescein diffusion is performed by epifluorescence microscopy or hair cross sections. Hair shafts from each experimental condition are dipped into a fluorophore solution (fluorescein; Ex=498 nm/Em=517 nm). Then, the excess of fluorophore is eliminated by extensive wash steps with ultra-pure water and the hair shafts sampled, cryo-preserved in Cryomatrix (OCT), snap-frozen into liquid nitrogen and kept at −80° C. until analysis. Hair shaft cross-sections (perpendicular to the hair axis, sagittal view) of 3 μm of thickness are obtained using a cryostat (Leica) for the analysis. Fluorescent images are collected with an epi-fluorescent microscope (ThermoFisher, Evos M5000) and analyzed with ImageJ software (Schneider, 2012). Image collection for the different conditions is achieved using identical conditions of acquisition (40× objective).

The raw images are collected in a range of intensity of specific signal from 0 to 65535 and analyzed with ImageJ software. The maximum intensity of diffused fluorophore is obtained and quantified by the integration of the specific fluorescence signal among the axis of penetration (from cuticle to cortex).

Three (3) images per condition are used to quantify the fluorescence intensity levels (R.F.U.); the mean value and standard deviation are obtained. Data treatment and statistical analysis are achieved by using graphPad Prism (La Jolla, California, USA) and included in the annexed file (Annex1-OxiProteomeView-D06CB.xlsx).

An efficacy value (%) is obtained for the experimental groups accordingly to the following formula, reported in the annexed excel file.

$$Efficacy \% ( test group X ) = [ Fluorence intensity ( Stress ) - Fluorescence intensity ( test group ) ] / \\ \\ [ Fluorescence intensity ( Stress ) - Fluorescence intensity ( Control ) ] * 100$$

As references, the control group is considered at maximum efficiency (100%) and the stress group at minimum efficiency (at 0%):

TABLE 6

| Exp. Group | Mean ± SEM | Defense efficacy (%) |
|---|---|---|
| Control | 100 ± 1.04 **** | 100% |
| Dying | 233 ± 11.96 | 0% |
| Azuki 1% + Dying | 151 ± 8.31 *** | 62% |

p-Values are obtained by ANOVA variance analysis followed by Dunnett's post-hoc test for multi-comparisons in comparison with stress.
**** p-Value < 0.001;
*** p-Value < 0.001;
**p-Value < 0.01;
*p-Value < 0.05

### Example 11

### Ex Vivo Repairing Efficacy in African Hair Shafts

African hair shafts are stressed heating at 95° C. during 30 min. After the stress exposure hair shafts are treated with Azuki purified hydrolysate at 1% (v/V) (active ingredient solubilized in ultra-pure water) for 30 minutes.

In situ detection of fluorescein diffusion is performed by epifluorescence microscopy or hair cross sections.

Just after treatments, hair shafts from each experimental condition are dipped into a fluorophore solution (fluorescein; Ex=498 nm/Em=517 nm). Then, the excess of fluorophore is eliminated by extensive wash steps with ultra-pure water and the hair shafts sampled, cryo-preserved in Cryomatrix (OCT), snap-frozen into liquid nitrogen and kept at −80° C. until analysis. Hair shaft cross-sections (perpendicular to the hair axis, sagittal view) of 3 μm of thickness are obtained using a cryostat (Leica) for the analysis. Fluorescent images are collected with an epi-fluorescent microscope (ThermoFisher, Evos M5000) and analyzed with ImageJ software (Schneider, 2012). Image collection for the different conditions is achieved using identical conditions of acquisition (40× objective).

The raw images are collected in a range of intensity of specific signal from 0 to 65535 and analyzed with ImageJ software. The maximum intensity of diffused fluorophore is obtained and quantified by the integration of the specific fluorescence signal among the axis of penetration (from cuticle to cortex). Three (3) images per condition are used to quantify the fluorescence intensity levels (R.F.U.); the mean value and standard deviation are obtained. Data treatment and statistical analysis are achieved by using graphPad Prism (La Jolla, California, USA)

An efficacy value (%) is obtained for the experimental groups accordingly to the following formula:

$$Efficacy \% ( test group X ) = [ Fluorence intensity ( Stress ) - Fluorescence intensity ( test group ) ] / \\ \\ [ Fluorescence intensity ( Stress ) - Fluorescence intensity ( Control ) ] * 100$$

The control group is considered at maximum efficiency (100%) and the stress group at minimum efficiency (at 0%):

TABLE 7

| Exp. Group | Mean ± SEM | Repair efficacy (%) |
|---|---|---|
| Control | 100 ± 11.0* | 100% |
| Heating | 212 ± 26.3 | 0% |
| Azuki 1% (v/v) + Heating | 114 ± 24.5* | 87% |

p-Values are obtained by ANOVA variance analysis followed by Dunnett's post-hoc test for multi-comparisons in comparison with stress.
*p-Value < 0.05

### Example 12

### Protection and Defense Against Lipid Peroxidation in Hair Samples

An aqueous solution of purified hydrolysate according to Example 3 is prepared at 2% (v/v in water) (“active ingredient”).

For protection test: 300 mg of hair are weighted in Flacon Tubes (Test) and 3 mL of active ingredient are added and left on an orbital shaker for 2 h. After the incubation time with the active ingredient, the hair is dried on cellulose paper for 30 min. After drying, 150 mg of Treated Hair are weighted in Petri Dishes and placed in a solar simulator for irradiation.

For protection and defense test: 300 mg of hair are weighted in Flacon Tubes (Test) and 3 mL active ingredient are added and left on an orbital shaker for 1 h 30 min. After the incubation time with the active ingredient, the hair is dried on cellulose paper for 30 min. Once samples are dried, 150 mg of Treated Hair are weighted in Falcon tubes and 1.5 mL of active ingredient added again thereby being also present during the irradiation treatment. The solution is vortexed and all the contents (active ingredient+Hair) are transferred to a Petri dish for solar Irradiation.

For defense test: 150 mg of untreated Hair are weighted in Falcon tubes and 1.5 mL of active ingredient added thereby being present during the irradiation treatment. The solution is vortexed and all the contents (active ingredient+Hair) are transferred to a Petri dish for solar Irradiation.

For control stress samples (untreated with active ingredient but irradiated hair), 150 mg of hair are weighed on Petri dishes for solar irradiation.

All samples are irradiated in a solar simulator (Solar simulator SOL500 Honle™, 4-5 mW/cm²) for 2 h.

Sample preparation for HPLC Analysis: Samples from the protection test are directly used for HPLC analysis whereas samples of the “protection and defense”; and “defense” tests, are dried on cellulose paper for 30 min. 15-30 mg of hair samples are weighted and 1 mL of MeOH is added and sonicated for 15 min. After sonication, in 10 mL glass tubes, the following reagents are added: 500 μL sample (extracted previously in MeOH), 750 μL H₃PO₄, 250 μL 2-Thiobarbituric Acid Solution at 42 mM in NaOH 1N and 450 μL H₂O-MilliQ. Tubes are vortexed and incubated at 90° C. for 60 min. Subsequently, 1.95 mL of Alkaline MeOH are added and vortexed before the HPLC analysis. A Kromasil™ 100 C-18, 5 μm, 25×4.5 cm columns is used with a High Performance Liquid Chromatography (Shimadzu): an isocratic method of 40% B during 15 min (A: 50 mM Buffer Phosphate pH 6.8, B: MeOH), 1 mL/min, 50 μL is injected at 36° C. The detector use is a fluorescence detector at λex=527 nm and λem=551 nm.

TABLE 8A
PROTECTION

| | CONTROL | 2 h UV | 2% AZUKI |
|---|---|---|---|
| MEAN | 44.8 | 100.0 | 69.8 |
| SEM | 1.0 | 4.9 | 3.9 |
| P VALUE | | | <0.0001 |
| % EFFICACY | | | 30.2. |

TABLE 8B
PROTECTION + DEFENSE

| | CONTROL | 2 h UV | 2% AZUKI |
|---|---|---|---|
| MEAN | 48.6 | 100.0 | 63.9 |
| SEM | 8.4 | 7.8 | 9.5 |
| P VALUE | | | <0.0001 |
| % EFFICACY | | | 36.1 |

TABLE 8C
DEFENSE

| | CONTROL | 2 h UV | 2% AZUKI |
|---|---|---|---|
| MEAN | 54.4 | 100.0 | 82.3 |
| SEM | 2.7 | 7.5 | 7.0 |
| P VALUE | | | 0.0181 |
| % EFFICACY | | | 17.7 |

### Example 13

### Human Dermal Fibroblasts and Human Epidermal Keratinocytes Cells (In Vitro), Protection and Defense Evaluation.

Human Dermal Fibroblasts (HDFa) and Human Epidermal Keratinocytes (HEKa) are co-cultivated in supplemented M106 medium and supplemented KGM2, respectively, at 37° C. in 5% CO₂ humidified air. Next day, cells are treated with Azuki hydrolysate of Example 3 at 1% (v/v) in culture medium for 1 hour at 37° C. in 5% CO₂ humidified air. After the protection treatment, fresh culture medium is supplemented with Azuki hydrolysate of Example 3 at 1% (v/v) and cells are stressed with Particle Matters at 2.34 μg/cm² (Diesel particulate matters NIST1650B) followed by solar irradiation (Solar simulator SOL500 Honle™, 32.4-36 J/cm²). Irradiation treatment is for 120 minutes. After solar irradiation, cells are incubated at 38° C. in 20% CO₂ humidified air for 2 hours. Just after stress exposure cell viability is measured using PrestoBlue™ reagent.

### Analysis of Cell Viability:

Resazurin, the active ingredient of PrestoBlue™ Reagent, is a nontoxic, cell-permeable compound that is virtually non-fluorescent. On entering live cells, the cellular reducing environment reduces resazurin to resorufin, a compound that is highly fluorescent. Viable cells continuously convert resazurin to resorufin, increasing the overall fluorescence of the media surrounding the cells, being directly proportional to the number of viable cells in each well.

Briefly, medium is aspirated and 100 μl/well of a 1/20 dilution of PrestoBlue™ in 1×DPBS is added. Then plates are incubated at 37° C. in 5% CO₂ humidified air for 10 min, protected from light. The resulting fluorescence is read at excitation wavelength=560 nm and emission wavelength=590 nm in a microtiter plate reader (Clariostar™, BMG LABTECH).

Each condition is tested at least in 5 replicates/wells and test items are assayed in 3 independent experiments.

TABLE 9

| % Viability | MEAN ± SEM | Protection efficacy (%) |
|---|---|---|
| CONTROL | 133.5 ± 4.75 **** | 100 |
| Non-treated + stress | 100.0 ± 3.44 | 0 |
| Azuki 1% + stress | 120.6 ± 4.73 ** | 61.53 |
| Azuki 2% + stress | 125.0 ± 4.58 **** | 74.51 |

p-Values are obtained by ANOVA variance analysis followed by Dunnett's post-hoc test for multi-comparisons in comparison with stress.
**** p-Value < 0.0001;
** p-Value < 0.01

### Example 14

### Human Dermal Fibroblasts and Human Epidermal Keratinocytes Cells (In Vitro), Repairing Evaluation.

Human Dermal Fibroblasts (HDFa) and Human Epidermal Keratinocytes (HEKa) are co-cultivated in supplemented M106 medium and supplemented KGM2, respectively, at 37° C. in 5% CO₂ humidified air. Next day, fresh culture medium is added, and cells are stressed with Particle Matters at 2.34 μg/cm² (Diesel particulate matters NIST1650B) followed by solar irradiation (Solar simulator SOL500 Honle, 24.3-36 J/cm², 90-120 minutes of irradiation). After solar irradiation, cells are incubated at 38° C. in 20% CO₂ humidified air for 22 hours. After the stress exposure, cells are washed and treated with azuki hydrolysate of Example 3 at 1% or 2% (v/v) in culture medium for 6 hours at 37° C. in 5% CO₂ humidified air.

Then, cell viability is measured using Prestoblue reagent as disclosed in Example 13.

TABLE 10

| Viability(%) | MEAN ± SEM | Repairing efficacy (%) |
|---|---|---|
| CTR | 142.6 ± 3.02 **** | 100 |
| Non-treated + stress | 100 ± 3.44 | 0 |
| Azuki 1% (v/v) + stress | 123.4 ± 2.24 **** | 54.78 |
| Azuki 2% (v/v) + stress | 116.9 ± 3.01 **** | 39.69 |

p-Values are obtained by ANOVA variance analysis followed by Dunnett's post-hoc test for multi-comparisons in comparison with stress.
**** p-Value < 0.0001

### Example 15

In Vitro Protection from DNA Oxidation in Human Epidermal Keratinocytes Cells

At the nuclear level, DNA is susceptible to oxidative damage. Of the four DNA bases, 2′-deoxyguanosine (dG) has the lowest ionization potential and thus is the most susceptible to oxidation. One of the positions where oxidation of dG can occur is at the C8 position, giving rise to 8-oxo-dG. 8-oxo-dG is a useful biomarker of oxidative damage in DNA. If not repaired, this lesion, and/or the products derived from it, may be misread by a polymerase during replication and lead to mispairing with A. Then, during the next round of replication, this A will correctly pair with T leading to a G-T transversion mutation. This replication sequence demonstrates the mutagenic nature of 8-oxo-dG and underscores the importance of protecting cells against oxidative DNA damage. [Edward Pelle, et al., “Ultraviolet-B-Induced Oxidative DNA Base Damage in Primary Normal Human Epidermal Keratinocytes and Inhibition by a Hydroxyl Radical Scavenger.” J. Invest. Dermatol. 2003]

Human Epidermal Keratinocytes (HEKa) are cultivated in supplemented KGM2 at 37° C. in 5% CO₂ humidified air. Then, cells are treated with azuki purified hydrolysate at 1% or 2% (v/v) in culture medium for 1 hour at 37° C. in 5% CO₂ humidified air. Then, cells together with the purified hydrolysates are incubated at high temperature (38° C.) and high levels of CO₂ (20% CO₂ humidified air) overnight to mimic the conditions of a “greenhouse effect”. Fresh culture medium is added and cells are stressed with Particle Matters at 1.58 μg/cm² (Diesel particulate matters NIST1650B) followed by solar irradiation (Solar simulator SOL500 Honle, 12.15-36 J/cm², 45-120 minutes of irradiation). After the stress exposure, the DNA extraction is carried out in Qiacube™ with the kit “DNeasy Blood & Tissue Kit” and DNA quantification is performed using Nanodrop™.

### Enzymatic Hydrolysis

DNA samples are digested to nucleosides using enzymatic hydrolysis. For each sample, 7 μL 15 mM 8-HQ (8-Hydroxyquinoline), and 50 μL buffer solution (50 mM ammonium acetate, 0.2 mM ZnCl2, pH 5.3) are added. Then 3 μL of Nuclease P1 (0.39 unit/μL) and 4 μL of alkaline phosphatase (1 unit/μL in 10 mM Tris pH 7.4) are added, and the solution is incubated at 37° C. for 60 min. The solution is centrifuged for 5 min at 14,000 rpm before LC-MS/MS analysis.

### HPLC-MS/MS Analysis

The HPLC-ESI-MS/MS system is a SHIMADZU HPLC model Nexera™ XR in line with a SHIMADZU triple quadrupole mass spectrometer, model LCMS-8040, equipped with an electrospray ionization source. Nitrogen is used as the drying and nebulizing gas. Argon is used as the collision gas for CID.

An InfinityLab Poroshell™ 120-EC-C18 column (4.6×75 mm×2.7 μm) are used at a flow rate of 0.5 mL/min, maintained at 36° C. A gradient solvent system from water+0.1% Acetic Acid (solvent A) to methanol (solvent B) is used for the separation. The gradient conditions are: 0-9 min 5% B-90% B, 9-12 min 90% B, 12.1-17 min 5% B. Analytes are quantified using tandem electrospray ionization mass spectrometry in positive-ion mode. Product ions are monitored in multiple-reaction monitoring mode.

Mass transition for 8-oxodG is m/z 284.15>168.05 and for 8-Hydroxyguanosine m/z 300.00>168.05. A secondary transition (m/z 284.15>140.1 and m/z 300.00>140.05, respectively) is used to confirm the identity of 8-oxodG and 8-Hydroxyguanosine respectively in samples, which are biomarkers of DNA oxidation. Injection volume is 25 uL.

TABLE 11A
8-oxodG (%)

| | MEAN ± SEM |
|---|---|
| Control | 60.70 ± 8.08** |
| Non-treated + stress | 100.0 ± 8.90 |
| Azuki 1% (v/v) + stress | 67.28 ± 7.52* |
| Azuki 2% (v/v) + stress | 53.67 ± 2.90*** |

p-Values are obtained by ANOVA variance analysis followed by Dunnett's post-hoc test for multi-comparisons in comparison with stress.
***p-Value < 0.001;
**p-Value < 0.01;
*p-Value < 0.05

TABLE 11B
8-Hydroxiguanosine (%)

| | MEAN ± SEM |
|---|---|
| Control | 41.23 ± 5.90*** |
| Non-treated + stress | 100.0 ± 6.05 |
| Azuki 1% + stress | 79.15 ± 5.57* |
| Azuki 2% + stress | 65.25 ± 3.19*** |

p-Values are obtained by ANOVA variance analysis followed by Dunnett's post-hoc test for multi-comparisons in comparison with stress.
****p-Value < 0.0001;
***p-Value < 0.001;
**p-Value < 0.01;
*p-Value < 0.05.

Both biomarkers of DNA oxidation (8-oxo-dG and 8-Hydroxiguanosine) are reduced after treatment with the purified hydrolysate compared to non-treated stressed cells, demonstrating the protective effect of the purified hydrolysate.

### Example 16

Antioxidant Activity in Combination with Other Ingredients

Suitable preservative [1% (w/w) Dermosoft™ 700B (INCI: Levulinic Acid (and) Sodium Levulinate (and) Glycerin (and) Aqua), 4% (w/w) Leucidal™ SF Max from Active Micro Technologies (AMT) (INCI: Lactobacillus Ferment) or 4% (w/w) Leucidal™ Liquid (INCI: Leuconostoc/Radish Root Ferment Filtrate)] is added onto deionized water under continuous stirring. Purified hydrolysate according to Example 3 is added on top of this solution to obtain a concentration of 44-88% (v/v), depending on sample dry weight. Final concentration of the purified protein hydrolysate (INCI: Phaseolus angularis Seed Extract) is then kept constant at 2.2 g per Kg of solution (0.22% (w/w)). Once the resulting solution becomes completely homogeneous, pH is adjusted with citric acid to a value of 4.5-5.0. Samples are maintained for 3 months at 40° C. or at room temperature and antioxidant activity determined by DPPH assay.

TABLE 12

| | % DPPH INHIBITION | | | |
|---|---|---|---|---|
| | | 3 M | | |
| | | | | room |
| | | | | temper- |
| | t = 0 | 40 °C | | ature |
| Purified azuki hydrolysate | 73.76 | Not tested | | Not |
| 1% (w/w) Dermosoft ™700B | 0 | | | tested |
| 0.5% (w/w) Sodium Benzoate | 0 | | | |
| Glycerin:H₂O | 0 | | | |
| 4% Leucidal ™ Liquid | 0 | | | |
| Purified azuki hydrolysate and | 86.0 | 71.8 | (−16.5) | 86.5 |
| 1%(w/w) Dermosoft ™ 700B + | | | | |
| 4% (w/w) Leucidal ™ SF Max | | | | |
| Purified azuki hydrolysate and 0.5% | 64.7 | 53.4 | (−17.4) | 61.6 |
| (w/w) Sodium Benzoate at low pH | | | | |
| Purified azuki hydrolysate and | 76.0 | 77.1 | (±1.4) | 78.3 |
| Glycerin:H₂O (80:20) | | | | |
| Purified azuki hydrolysate + 4% | 86.6 | 84.1 | (−2.9) | 87.0 |
| Leucidal ™ Liquid | | | | |

None of the preservatives demonstrated antioxidant activity. Combination with 4% Leucidal™ Liquid shown a synergistic effect between the purified hydrolysate and Leuconostoc/Radish Root Ferment in Leucidal™ liquid.

### Example 17

### Isolation and Identification of Active Peptides

The purified extract according to Example 3 is further fractionated by using semi-preparative high-performance liquid chromatography (HPLC) and antioxidant activity assayed by DPPH for identifying the active fraction or peptides.

Semipreparative HPLC (Shimadzu Corporation) is used with a LC-8A binary pump, a SCL-8A system controller, a SIL-8A automatic sample injector (20 mL loop), an SPD-6A UV wavelength detector, a C-R6A data recording module, and a fraction collector. Appropriate dilutions of purified hydrolysate in water are loaded into a reverse-phase C18 column (PuriFlash® PP-15C18-F0080, 31×205 mm, 15 μm; Interchim®). Peptides are separated using an eluent system consisting of eluents A (water/trifluoroacetic acid 1000:1) and B (acetonitrile/trifluoroacetic acid 1000:1). Elution is carried out at a flow of 35 ml/min using the following gradient composition: isocratic of 0% B in 3 min, 0 to 30% of B in 10 min, and 30 to 75% of B in 7 min, monitoring absorbance of eluting peaks at 220 nm.

Collected tubes are analyzed by analytical HPLC at 3.0 mg/ml using a Cortecs C18+column (4.6×50 mm; 2.7 μm; Waters®) at a flow of 1.6 ml/min using the following gradient composition: isocratic of 0% B in 2 min, 0 to 30% of B in 5 min, and 30 to 75% of B in 5 min, monitoring absorbance of eluting peaks at 220 nm. Different fractions are obtained from the purified hydrolysate and lyophilized for further analysis and separation. Fractions are reconstituted in water and their antioxidant activity is determined by measuring the percentage of DPPH inhibition using the methodology described above.

Fractions showing the highest DPPH activity are further sub-fractionated.

Peptides contained in the different fractions are dissolved in water at a concentration of 25 mg/ml and separated by loading 4 mL in the abovementioned semi-preparative HPLC system using the same PuriFlash column. Elution is performed at a flow of 35 ml/min using the following gradient composition: isocratic of 0% B in 3 min, and 5 to 15% of B in 15 min, monitoring absorbance of eluting peaks at 220 nm. Collected tubes are analyzed by means of similar analytical HPLC conditions as described above but using an isocratic gradient of 0% B in 2 min followed by 5 to 15% of B in 6 min. Different subfractions showing antioxidant activity are obtained.

### Identification of Antioxidant Peptides by High Resolution Mass Spectrometry

Subfraction are separated by reverse-phase nano-HPLC coupled to high resolution LTQ Orbitrap Velos mass spectrometer (Thermo Fisher Scientific). Peptide separation took place using a C18 nanocolumn (ID 75 μm, 15 cm, 3 μm particle diameter, Nikkyo Technos Co. LTD) connected to a trap nanocolumn (ID 100 μm, 2 cm, 5 μm particle diameter, Thermo Fisher Scientific). A 120 min gradient in the following phases: 0.1% formic acid/water (phase A), and 0.1% formic acid/acetonitrile (phase B) is used, at a flow rate 300 nL/min. Real time ionization and peptide fragmentation is achieved by acquiring FT spectra in a resolution of 30.000 FHMW) followed by FT-MS/MS scan and selecting the 10 most intense ions with HCD fragmentation, collision energy of 35% and dynamic exclusion of 0.5 min. All spectra are acquired in positive ion mode.

To identify peptide sequences, the Proteome Discoverer 1.4.0.288 program (Thermo) with MASCOT 2.5 is used. The identified peptides are individually synthesized by applying standard solid phase peptide synthesis, using Fmoc/tBu chemistry protocols. As starting resin, CTC-PS or HO-Trt-Chem Matrix® resin is used. In the latter case, initial resin chlorination took place by overnight reaction with 37% HCl in dry THF. The first amino acid is introduced as a solution in DMF in presence of DIEA as base, followed by capping of the resin with MeOH. The resin is washed with DMF, prior to removal of Fmoc group by treatment with a 20% solution of piperidine in DMF, followed by washings in the same solvent. Subsequent residues are incorporated in DMF employing DIPCDI and Oxyma as coupling reagents. After verification of reaction completion using ninhydrin test, the solvent is filtrated away by suction. After this, the resin is washed with DMF and the Fmoc-deprotection step is repeated as abovementioned or in 2% DBU in DMF. Coupling-deprotection cycles are repeated for introduction of the remaining amino acids, with increasing Fmoc removal treatment times from the 6^{th} residue. Oxyma (0.5M) is introduced in the cocktail whenever Asp residue is already incorporated into the growing peptide chain.

The peptidyl resin is washed and dried using DMF, DCM and diethyl ether and finally left under vacuum overnight to get rid of solvent traces. Cleavage of the crude product from the solid support and removal of the side-chain protecting groups is achieved by acidolysis using TFA and 5% water as scavenger. The peptide is precipitated out of the acidic mixture with Et₂O, followed by washes with the same solvent and drying under reduced pressure. Crude trifluoroacetate salt peptide is lyophilized and analyzed by analytical HPLC-MS, affording peptide purities over 80%.

Synthesized peptides are further evaluated for its capacity to inhibit DPPH as a reference for their antioxidant potential. The most active peptides as well as their antioxidant activity (DPPH) and presence in the purified hydrolysate are shown in Table 13. All synthesized sequences displayed the ability to inhibit DPPH, being SEQ. ID. NO 1, 2, 3, 4 and 5 the most antioxidant peptides found in the F′2 subfraction.

TABLE 13

| | | DPPH | |
|---|---|---|---|
| SEQ | | inhibition | Concentration |
| ID No. | Sequence | (%) | (ppm) |
| 1 | VAEQEQEQAKRQ | 78.7 | 0.023 |
| 2 | LPQQEQGHDR | 77.5 | 0.033 |
| 3 | VDAQPEEQQR | 100 | 0.024 |
| 4 | GINAENNQRN | 96.3 | 0.068 |
| 5 | EYTGDAAGR | 83.9 | 0.007 |

### Example 18

### In Vitro Proliferation Assay on Human Epidermal Keratinocyte Cells

Human Epidermal Keratinocyte Cells from adult (HEKa) are cultivated with Keratinocyte Growth Medium II (Promocell™) supplemented with Supplement Mix C39016-CaCl2 solution (Promocell™) at 37° C. and 5% CO₂. Cells are treated with purified hydrolysate of Example 3 at 1% (v/v) in culture medium. After 24 hours incubation, live cells are quantified with PrestoBlue™ Cell Viability Reagent (Thermo Fisher Scientific).

### Analysis of Cell Viability:

Resazurin, the active ingredient of PrestoBlue™ Reagent, is a nontoxic, cell-permeable compound that is virtually non-fluorescent. On entering live cells, the cellular reducing environment reduces resazurin to resorufin, a compound that is highly fluorescent. Viable cells continuously convert resazurin to resorufin, increasing the overall fluorescence of the media surrounding the cells, being directly proportional to the number of viable cells in each well.
Briefly, medium is aspirated and 100 ul/well of a 1/20 dilution of PrestoBlue™ in 1×DPBS is added. Then plates are incubated at 37° C. in 5% CO₂ humidified air for 10 min, protected from light. The resulting fluorescence is read at excitation wavelength=560 nm and emission wavelength=590 nm in a microtiter plate reader (Clariostar™, BMG LABTECH).

Each condition is tested at least in 3 replicates/wells and test items are assayed in 3 independent experiments.

TABLE 14

| | Cell viability % |
|---|---|
| Dose Tested | (MEAN ± SEM) |
| CONTROL | 100.00 ± 1.99 |
| Azuki 1% (v/v) | 127.62 ± 14.23 ** |

Statistical significance:
** p < 0.01, calculated using an unpaired Student's t test.

### Example 19

Lipid Peroxidation Repair from Solar Radiation (Rinse-Off)

Eight hundred milligrams (800 mg) of Asian hair are weighed in Petri Dishes and Irradiated in a Solar Simulator (Solar simulator SOL500 Honle, 32.4-36 J/cm²) for 2 h. Once the irradiation time is over, 600 mg of damaged hair is weighted in Flacon Tubes and 6 mL of azuki purified hydrolysate at 22 μg/ml are added and left on an orbital shaker for 30 min. After the incubation time with the azuki purified hydrolysate, the hair is rinsed with water at 30° C. for 30 seconds. Then, half of the hair is left to dry in cellulose paper and the remaining part (300 mg) is treated again with 3 mL azuki purified hydrolysate at 22 μg/mL. Samples are left on an orbital shaker for 30 min and then hair is rinsed with water at 30° C. for 30 seconds and left to dry until its analysis. Non-damaged (i.e., untreated) Asian hair is used as CONTROL sample. For damage control samples (“solar radiation damaged control”), damaged hair after Solar Irradiation application are used.

In Eppendorf tubes, 15-30 mg of hair samples are weighed, and 1 ml of methanol is added and sonicated for 15 min. After sonication, in 10 mL glass tubes, the following reagents are added: 500 μL sample (extracted previously in methanol), 750 μL H₃PO₄, 250 μL 2-Thiobarbituric Acid Solution at 42 mM in NaOH 1N and 450 μL H₂O-MilliQ. Tubes are vortexed and incubated at 90° C. for 60 min. Subsequently, 1.95 mL of alkaline methanol are added and vortexed before the HPLC analysis.

HPLC analysis is performed under the following conditions: Kromasil™ 100 C-18, 5 μm, 25×4.5 cm, an isocratic method at 40% B is used and mobile phases are A: 50 mM Buffer Phosphate pH 6.8 and B: MeOH, a fluorescence detector is used at Δex=527 nm and λem=551 nm.

### Results:

When azuki purified hydrolysate is applied once at 22 μg/mL in Solar Radiation Damaged Hair, there is an inhibition of Lipid Peroxidation by 35.6%, as it can be observed in FIG. 1 and Table 15.

TABLE 15

| | | 2 h Solar | 2 h Solar Radiation + 22 μg/mL |
|---|---|---|---|
| | Control | Radiation | azuki purified hydrolysate |
| MEAN | 44.8 | 100.0 | 64.4 |
| SEM | 1.0 | 5.3 | 2.8 |
| P Value | — | — | <0.001 |
| % Efficacy | — | — | 35.6 |

Therefore, results demonstrate a protective effect of azuki purified hydrolysate against solar radiation.

### Example 20

### Leave-On Serum

Leave-on serum is prepared including the following ingredients:

TABLE 16

| Ingredient | | wt % |
|---|---|---|
| WATER | | 95.20 |
| DISSOLVINE NA2 P | | 0.20 |
| Carbopol ® Ultrez ™ 21 polymer | | 0.35 |
| Active ingredient | WATER (AQUA) | 1.96 |
| | Leucidal ™ liquid | 0.04 |
| | (LEUCONOSTOC/RADISH ROOT | |
| | FERMENT FILTRATE) | |
| | Azuki peptide hydrolysate | 0.004 |
| | (PHASEOLUS ANGULARIS SEED | |
| | EXTRACT) | |
| | Citric acid | 0.0004 |
| POLYSORBATE 20 | | 1.05 |
| BABY ALOE VERA (PR155) | | 0.20 |
| EUXYL PE 9010 (BC042) | | 0.50 |
| SODIUM HYDROXIDE 20% w/w | | 0.50 |

### Example 21

### Face Cream

Face cream is prepared including the following ingredients:

TABLE 17

| Ingredient | | wt % |
|---|---|---|
| WATER (AQUA) | | 74.24 |
| ZEMEA PROPANEDIOL | | 7.00 |
| SCHERCEMOL CO | | 5.00 |
| GLYCERIN | | 2.00 |
| GLUCAM E-20 HUMECANT | | 2.00 |
| CAPRYLIC CAPRIC | | 2.50 |
| TRIGLYCERIDES | | |
| NOVEMER EC-2 | | 2.00 |
| DERMOSOFT 700B | | 1.00 |
| CETIOL SB-45 | | 0.50 |
| DERMOSOFT GMCY | | 0.50 |
| CARBOPOL ™ ULTREZ 20 | | 0.30 |
| POLYMER | | |
| SODIUM HYDROXIDE 20% w/w | | 0.96 |
| Active ingredient | WATER (AQUA) | 1.96 |
| | Leucidal ™ liquid | 0.04 |
| | (LEUCONOSTOC/RADISH ROOT | |
| | FERMENT FILTRATE) | |
| | Azuki peptide hydrolysate | 0.004 |
| | (PHASEOLUS ANGULARIS SEED | |
| | EXTRACT) | |
| | Citric acid | 0.0004 |

### Example 22

### In Vivo Efficacy in Hair Protection Against Exposome's Real Outdoor Conditions

The study is carried out for 14 days. Sixty-two (62) female volunteers aged between 19 and 49 years old are included in the study. Volunteers included in the study had different hair types according to Andre Walker Hair Typing System and at least 50% of them had their hair dyed. Subjects are divided into two groups. One of the groups applied the leave-on serum described in Example 20 (“Active leave-on serum”) and the other applied a placebo leave-on serum having the same composition except azuki hydrolysate. Regardless of the group, the leave-on serum is applied for 14 days on the whole hair daily. During the study length, all subjects are required to spend daily 4 hours outdoors to expose hair to real outdoor conditions. Subjects served as their own reference and results obtained at 14 days are compared with those obtained at initial time.

Volunteers' hair is clinically evaluated by an expert at initial time and after 14 days of product application. The efficacy of the product against exposome's real outdoor conditions is assessed by:
Volunteers' hair split ends are evaluated by an expert who performed a clinical evaluation grading split ends through a scale at initial time and 14 days of product application.

TABLE 18
Mean split ends variation of active and placebo leave-on
serums after 14 days of product application.

| | Mean split ends variation (%) | |
|---|---|---|
| | Active leave-on | Placebo leave-on |
| | serum | serum |
| [(T14 days − T0)/T0] × 100 | −12.9 * | −7.4 |

* p < 0.05 calculated using paired Wilcoxon test.

Results demonstrate that after 14 days of application of the active leave-on serum there is a significant decrease in split ends with respect to initial time.

Volunteers' hair fiber alignment is evaluated by an expert who performed a clinical evaluation grading fiber alignment through a scale at initial time and 14 days of product application.

TABLE 19
Average fiber alignment variation of active and placebo
leave-on serums after 14 days of product application.

| | Average fiber alignment variation (%) | |
|---|---|---|
| | Active leave-on | Placebo leave-on |
| | serum | serum |
| [(T14 days − T0)/T0] × 100 | 22.2 ** | 16.1 |

** p < 0.01 calculated using paired Wilcoxon test.

Results demonstrate that after 14 days of application of the active leave-on serum there is a significant increase in fiber alignment with respect to initial time.
Volunteers' hair frizz is evaluated by an expert who performed a clinical evaluation grading frizz through a scale at initial time and 14 days of product application.

TABLE 20
Average frizz variation of active and placebo leave-on
serums after 14 days of product application.

| | Average frizz variation (%) | |
|---|---|---|
| | Active leave-on | Placebo leave-on |
| | serum | serum |
| [(T14 days − T0)/T0] × 100 | −13.6 ** | −9.4 * |

** p < 0.01 and * p < 0.05 calculated using paired Wilcoxon test.

Results demonstrate that after 14 days of application of active leave-on serum there is a significant decrease in frizz with respect to initial time.

### Example 23

### In Vivo Efficacy in Hair Protection Against Exposome's Real Outdoor Conditions in Hair Type 3 and 4.

The study is carried out for 14 days. Twenty-one (21) female volunteers aged between 23 and 44 years old are included in the study. Volunteers included in the study had hair types 3 (hair which is curly) and 4 (hair which is coiled) according to the Andre Walker Hair Typing System and at least 50% of them had their hair dyed. Subjects are divided into two groups. One of the groups applied the leave-on serum described in example 20 (“active leave-on serum”) and the other applied a placebo leave-on serum. Regardless of the group, the leave-on serum is applied for 14 days on the whole hair daily. During the study length, all subjects are required to spend daily 4 hours outdoors to expose hair to real outdoor conditions. Subjects served as their own reference and results obtained at 14 days are compared with those obtained at initial time.

Volunteers' hair is clinically evaluated by an expert at initial time and after 14 days of product application. The efficacy of the product against exposome's real outdoor conditions is assessed by volunteers' hair volume variation.

Hair volume is evaluated by an expert who performed a clinical evaluation grading hair volume through a scale at initial time and 14 days of product application.

TABLE 21
Mean hair volume variation of active and placebo leave-on
serums after 14 days of product application.

| | Mean hair volume variation (%) | |
|---|---|---|
| | Active leave-on | Placebo leave-on |
| | serum | serum |
| [(T14 days − T0)/T0] × 100 | −19.9 (ls) | −13.6 |

(ls) p < 0.1 calculated using paired Wilcoxon test.

Results demonstrate that after 14 days of application of the active leave on serum there is a significant decrease in hair volume with respect to initial time in volunteers with hair type 3 and 4.

### Example 24

### In Vivo Efficacy in Skin Protection Against Exposome's Real Outdoor Conditions in Multiethnic Skin Type Volunteers.

The study is carried out for 28 days. Sixty-two (62) female volunteers of multiple ethnicities aged between 19 and 49 years old are included in the study. Subjects are divided into two groups. One of the groups applied the composition described in example 21 (“active cream”) on the other applied a placebo cream having the same composition except azuki protein hydrolysate. Regardless of the group, the cream is applied for 28 days on the whole face twice a day at morning and afternoon. During the study length, all subjects are required to spend daily 4 hours outdoors to expose skin to real outdoor conditions. Subjects served as their own reference and results obtained at different study times are compared with those obtained at initial time. The efficacy of the product against exposome's real outdoor conditions is assessed by:
Barrier function protection assessment by trans epidermal water loss (TEWL): When the barrier function of the skin is damaged an increase in TEWL can be expected. Tewameter® measurements on cheek are carried out at initial time, 14 days and 28 days of product application.

TABLE 22
TEWL variation of active and placebo creams
after 14 and 28 days of product application.

TEWL variation (%)

| | Active cream | Placebo cream |
|---|---|---|
| [(T14 days − T0)/T0] × 100 | 0.1 | 19.8 |
| [(T28 days − T0)/T0] × 100 | −0.8 | 12.2 |

Significance calculated using Wilcoxon paired test of active versus placebo cream at 14 days of application is * p < 0.05.

Results demonstrate that after 14 days of application of active cream there is a significant decrease in TEWL with respect to placebo cream.
Skin texture heterogeneity is assessed by the average perimeter of spots in volunteers' cheek. Images at initial time and 28 days of product application are acquired with Colorface® and perimeter average of spots in Region Of Interest (ROI) is measured with Framescan™ software.

TABLE 23
Spot perimeter average variation of active and placebo
creams after 28 days of product application.

| | Spot perimeter average variation (%) | |
|---|---|---|
| | Active cream | Placebo cream |
| [(T28 days − T0)/T0] × 100 | −3.2 * | −0.2 |

* p < 0.05 calculated using a t-student test.

Results demonstrate that after 28 days of application of active cream there is a significant decrease in spot perimeter with respect to initial time, leading to a decrease in skin texture heterogeneity.
Skin tone homogeneity is evaluated by a dermatologist who performed a clinical evaluation grading skin's tone homogeneity through a scale at initial and 28 days of product application.

TABLE 24
Skin tone homogeneity average variation of active and
placebo creams after 28 days of product application.

Skin tone homogeneity
average variation (%)

| | Active cream | Placebo cream |
|---|---|---|
| [(T28 days − T0)/T0] × 100 | 12.5 * | 6.7 |

* p < 0.05 calculated using a t-student test.

Results demonstrate that after 28 days of application of active cream there is a significant increase in skin's tone homogeneity according to the expert evaluation with respect to initial time.

### Example 25

### In Vivo Efficacy in Skin Protection Against Exposome's Real Outdoor Conditions in Asian Ethnic Volunteers.

The study is carried out for 28 days. Seven (7) female volunteers of Asian ethnicity aged between 23 and 49 years old are included in the study. Four (4) subjects applied active cream and three (3) placebo cream for 28 days on the whole face twice a day in the morning and afternoon. Subjects applying active cream applied the composition described in example 21 and the subjects applying placebo cream applied the same composition except azuki peptide hydrolysate. During the study length, all subjects are required to spend daily 4 hours outdoors to expose skin to real outdoor conditions. Subjects served as their own reference and results obtained at 28 days are compared with those obtained at initial time.

Volunteers' images of forehead are acquired at initial time and 28 days of product application with Colorface®. The efficacy of the product against exposome's real outdoor conditions in Asian volunteers is assessed by different parameters obtained from image analysis:
Skin erythema is assessed by the average variation of a* parameter which is used for measuring color related to erythema.

TABLE 25
a * parameter average variation of active and placebo
creams after 28 days of product application.

| | a * parameter average variation (%) | |
|---|---|---|
| | Active cream | Placebo cream |
| [(T28 days − T0)/T0] × 100 | −8.3 * | −1.7 |

* p < 0.05 calculated using paired t-student test.

Skin melanin is evaluated by the average variation b* parameter which is used for measuring color related to melanin.

TABLE 26
b* parameter average variation of active and placebo
creams after 28 days of product application.

| | b* parameter average variation (%) | |
|---|---|---|
| | Active cream | Placebo cream |
| [(T28 days − T0)/T0] × 100 | −10.5 (ls) | −2.7 |

p < 0.1 is calculated using paired t-student test.

Results demonstrate that after 28 days of application of active there is a significant decrease in a* and b* parameters with respect to placebo cream in Asian volunteers. According to this, skin erythema and skin melanin decreases after 28 day product application in Asian volunteers. These parameters are related with a visual reduction of skin redness and an improving of skin brightness, respectively.

### Example 26

### In Vivo Efficacy in Skin Protection Against Exposome's Real Outdoor Conditions in Caucasian Ethnic Volunteers.

The study is carried out for 28 days. Twenty-one (21) female volunteers of Caucasian ethnicity aged between 19 and 39 years old are included in the study. Ten (10) subjects applied active cream and eleven (11) placebo cream for 28 days on the whole face twice a day in the morning and afternoon. Subjects applying active cream applied the composition described in example 21 and the subjects applying placebo cream having the same composition except azuki hydrolysate. During the study length, all subjects are required to spend daily 4 hours outdoors to expose skin to real outdoor conditions. Subjects served as their own reference and results obtained at 28 days are compared with those obtained at initial time. Volunteers' images of cheeks are acquired at initial time and 28 days of product application with Colorface®. The efficacy of the product against exposome's real outdoor conditions in Caucasian volunteers is assessed by a* parameter obtained from image analysis.

Skin erythema is evaluated at initial time and after 28 days of product application. Skin erythema is assessed by the mean variation of a* parameter which is used for measuring color related to erythema.

TABLE 27
a * parameter mean variation of active and placebo
creams after 28 days of product application.

| | a * parameter mean variation (%) | |
|---|---|---|
| | Active cream | Placebo cream |
| [(T28 days − T0)/T0] × 100 | −4.5 * | −0.5 |

* p < 0.05 calculated using paired t-student test.

Results show that after 28 days of product application there is a significant decrease in a* parameter with respect to placebo cream in Caucasian volunteers leading to a skin showing less erythema (i.e., skin redness).

### Example 27

### In Vivo Efficacy in Skin Protection Against Exposome's Real Outdoor Conditions in African Ethnic Volunteers.

The study is carried out for 28 days. Nineteen (19) female volunteers of African ethnicity aged between 23 and 44 years old are included in the study. Nine (9) subjects applied active cream and ten (10) placebo cream for 28 days on the whole face twice a day in the morning and afternoon. Subjects applying active cream applied the composition described in example 21 and the subjects applying placebo cream applied the same composition except azuki hydrolysate. During the study length, all subjects are required to spend daily 4 hours outdoors to expose skin to real outdoor conditions. Subjects served as their own reference and results obtained at 28 days are compared with those obtained at initial time.

Volunteers' images of cheeks are acquired at initial time and 28 days of product application with Colorface®. The efficacy of the product against exposome's real outdoor conditions in African volunteers is assessed by b* parameter obtained from image analysis.

Skin melanin is evaluated at initial time and after 28 days of product application. Skin melanin is assessed by the average variation of b* parameter which is used for measuring color related to melanin.

TABLE 28
b * parameter average variation of active and placebo
creams after 28 days of product application.

| | b * parameter average variation (%) | |
|---|---|---|
| | Active cream | Placebo cream |
| [(T28 days − T0)/T0] × 100 | −4.6 * | −1.3 |

* p < 0.05 calculated using paired t-student test.

Results demonstrate that after 28 days of product application there is a significant decrease in b* parameter with respect to placebo cream in African volunteers leading to a skin with less melanin (i.e., higher brightness).

A Sequence Listing in computer-readable xml form is incorporated herein by reference. The name of the file is P209501WO.xml, the date of creation was Sep. 12, 2024, and the size of the file is 5338 bytes.

1-56. (canceled)

57. A protein hydrolysate of azuki beans comprising at least one compound selected from the group consisting of:
(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-GIn-Gly-His-Asp-Arg;
(iii)
(SEQ ID No: 3)
Val-Asp-Ala-Gln-Pro-Glu-Glu-Gln-Gln-Arg;
and
(iv)
(SEQ ID No: 4)
Gly-Ile-Asn-Ala-Glu-Asn-Asn-Gln-Arg-Asn.

58. The protein hydrolysate of azuki beans according to claim 1 comprising:
(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-GIn-Gly-His-Asp-Arg;
(iii)
(SEQ ID No: 3)
Val-Asp-Ala-Gln-Pro-Glu-Glu-Gln-Gln-Arg;
and
(iv)
(SEQ ID No: 4)
Gly-Ile-Asn-Ala-Glu-Asn-Asn-Gln-Arg-Asn.

59. The protein hydrolysate of azuki beans according to claim 1 further comprising:
(v)
(SEQ ID No: 5)
Glu-Tyr-Thr-Gly-Asp-Ala-Ala-Gly-Arg.

60. The protein hydrolysate according to claim 1, wherein said hydrolysate does not contain any components having a molecular weight greater than 3 kDa.

61. The protein hydrolysate according to claim 1, wherein, when present, each of the following compounds is present in an amount of from 0.001 to 0.15 ppm:
(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-GIn-Gly-His-Asp-Arg;
(iii)
(SEQ ID No: 3)
Val-Asp-Ala-Gln-Pro-Glu-Glu-Gln-Gln-Arg;
and
(iv)
(SEQ ID No: 4)
Gly-Ile-Asn-Ala-Glu-Asn-Asn-Gln-Arg-Asn.

62. The protein hydrolysate according to claim 1, further comprising at least 0.05 mg of polyphenols per milliliter of protein hydrolysate as determined by the Folin-Ciocalteu method.

63. The protein hydrolysate according to claim 1, wherein said hydrolysate has been obtained by enzymatically hydrolyzing protein of azuki beans in the presence of a serine endoprotease, wherein the serine endoprotease is subtilisin A.

64. The protein hydrolysate according to claim 1, wherein the hydrolysate has been obtained by a method comprising:
a) providing a suspension of ground azuki beans in water;
b) treating the suspension with alkali to solubilize proteins in the ground azuki beans;
c) enzymatically treating the suspension obtained in b) with a serine endoprotease;
d) deactivating the serine endoprotease in the suspension obtained in c) to form a crude hydrolysate; and
e) removing components from the crude hydrolysate having a molecular weight of greater than 3 KDa, wherein e) comprises:
i. recovering a supernatant from the crude hydrolysate by solid/liquid separation; and
ii. removing components from the supernatant having a molecular weight of greater than 3 KDa.

65. The protein hydrolysate according to claim 64, wherein b) comprises adjusting the pH of the suspension to a pH of from 6.5 to 7.5.

66. The protein hydrolysate according to claim 64, wherein c) is carried out at a temperature of from 20 to 30° C. and wherein d) comprises heating the suspension obtained in c) to a temperature of at least 70° C.

67. The protein hydrolysate according to claim 1, further comprising a filtrate obtained by the fermentation of Raphanus sativus root by the microorganism Leuconostoc.

68. A personal care or cosmetic composition comprising:
a protein hydrolysate of azuki beans according to claim 1; and
at least one cosmetically acceptable excipient or ingredient.

69. The personal care or cosmetic composition according to claim 68, wherein the protein hydrolysate of azuki beans is in an amount of from 0.001 wt % to 10 wt %, based on the total weight of the personal care or cosmetic composition.

70. A method of treatment and/or care of the skin and/or hair comprising topically administering a protein hydrolysate of azuki beans according to claim 1 to a subject, wherein the treatment and/or care:
hinders or delays the adverse effects of the exposome on the skin and/or hair;
hinders or delays the adverse effects of air pollution containing particulate matter on the skin and/or hair;
hinders or delays the adverse effects of sunlight on the skin and/or hair;
hinders or delays the adverse effects of dyeing on hair; and/or
hinders or delays the physical barrier function of the skin.

71. A compound selected from the group consisting of:
(i)
(SEQ ID No: 1)
Val-Ala-Glu-Gln-Glu-Gln-Glu-Gln-Ala-Lys-Arg-Gln;
(ii)
(SEQ ID No: 2)
Leu-Pro-Gln-Gln-Glu-Gln-Gly-His-Asp-Arg;
(iii)
(SEQ ID No: 3)
Val-Asp-Ala-Gln-Pro-Glu-Glu-Gln-Gln-Arg;
(iv)
(SEQ ID No: 4)
Gly-Ile-Asn-Ala-Glu-Asn-Asn-Gln-Arg-Asn;
(v)
(SEQ ID No: 5)
Glu-Tyr-Thr-Gly-Asp-Ala-Ala-Gly-Arg,
and
stereoisomers, and cosmetically acceptable salts thereof.

72. A process for preparing a protein hydrolysate of azuki beans, comprising:
a) providing a suspension of ground azuki beans in water;
b) treating the suspension with alkali to solubilize proteins in the ground azuki beans;
c) enzymatically treating the suspension obtained in b) with a serine endoprotease;
d) deactivating the serine endoprotease in the suspension obtained in c) to form a crude hydrolysate; and
e) removing components from the crude hydrolysate having a molecular weight of greater than 3 KDa, wherein e) comprises:
i. recovering a supernatant from the crude hydrolysate by solid/liquid separation; and
ii. removing components from the supernatant having a molecular weight of greater than 3 KDa.

73. The process according to claim 72, wherein the serine endoprotease is subtilisin A.

74. The process according to claim 72, wherein b) comprises adjusting the pH of the suspension to a pH of from 6.5 to 7.5.

75. The process according to claim 72, wherein c) is carried out at a temperature of from 20 to 30° C. for a period of from 30 to 120 minutes and wherein d) comprises heating the suspension obtained in c) to a temperature of at least 70° C. for at least 20 minutes.

76. The process according to claim 72, wherein a percentage of protein in the ground azuki beans is from 15 to 25% w/w and wherein the ground azuki beans in the suspension of ground azuki beans in water is present in an amount of from 20 to 100 g per Kg of water.
